(19) United States
(12) Patent Application Publication
(10) Pub. No.: US 2005/0153943 A1
(43) Pub. Date: Jul. 14, 2005

(54) Methods of simultaneously treating mucositis and fungal infection

(76) Inventor: Robert A. Ashley, Tucson, AZ (US)

Correspondence Address: Ronald J Baron;Hoffmann & Baron, 6900 Jericho Turnpike, Syosset, NY, 11791, US

(21) Appl. No.: 10/508,839

(22) PCT Filed: May 6, 2003
(86) PCT No.: PCT/US03/14291

Related U.S. Application Data
(60) Provisional application No. 60/377,998, filed on May 6, 2002.

(51) Int. Cl. A61K 31/65

(52) U.S. Cl. 514/152

(57) ABSTRACT

A method for simultaneously treating mucositis and fungal infection in a mammal in need thereof, said method comprising administering to said mammal an effective amount of an anti-mucositis and anti-fungal pharmaceutical composition consisting of a tetracycline compound in an amount that is effective to simultaneously treat mucositis and fungal infection, but has substantially no antibiotic activity.

The present application claims benefit of U.S. provisional application Ser. No. 60/377,998, filed May 6, 2002, which is incorporated herein by reference.

### BACKGROUND OF INVENTION

Mucositis is a disease characterized by inflammation of the mucosa and destruction of the mucosa epithelium. Such destruction results in erythema, ulcerations and severe pain.

Mucositis often arises in mammals that have compromised immune systems. For example, mucositis often appears as a complication of antineoplastic therapy, such as cancer chemotherapy and/or radiation therapy.

Fungal growth is also seen in patients whose immune systems have been compromised, such as AIDS patients or chemotherapy patients. Fungal growth often accompanies mucositis.

Methods for treating mucositis have been disclosed. For example, Sonis et al. have disclosed the use of inflammatory cytokine inhibitors, MMP inhibitors and/or mast cell inhibitors to treat mucositis. (International PCT application WO 99/45910.) Examples of MMP inhibitors are said to include tetracyclines, such as minocycline, tetracycline HCl, and doxycycline. Sonis et al. state that it is preferred to include an “antimicrobial agent” in their treatment. The only reason given by Sonis et al. for adding an antimicrobial agent is that the presence of bacteria leads to secondary infections and amplified tissue damage. Sonis et al. neither mention, nor suggest, including anti-fungal agents.

Lawter et al. acknowledge the disclosure by Sonis et al. of the use of MMP inhibitors to treat mucositis. (International PCT application WO 01/19362.) According to Lawter et al., the only MMP inhibitors which appear to significantly reduce the symptoms of the mucositis are the tetracyclines. They attempt to reduce side effects by using a tetracycline that is poorly absorbed from the gastro-intestinal tract. A tetracycline is defined as being poorly absorbed from the gastrointestinal tract if it has a bioavailability of about 10% or less. Lawter et al. describe fungi as not being susceptible to tetracyclines. Accordingly, Lawter et al. disclose that their formulation may optionally contain an anti-fungal agent.

Antibiotics, such as tetracyclines, have long been considered ineffective as anti-fungal agents. (Lu et al., Journal of Dental Research, AADR Abstracts, 80:141, No. 845, (January 2001).) Nevertheless, Lu et al. tested the effects of two chemically modified non-antibiotic tetracyclines, 6-demethyl-6-deoxy-4-de(dimethylamino)tetracycline (CMT-3) and 6-α-deoxy-5-hydroxy-4-de(dimethylamino)tetracycline (CMT-8), in vitro against eleven different species of fungi. CMT-3 showed anti-fungal activity with eight of the eleven species. However, CMT-8 was said to show weak or no anti-fungal activity.

Most current anti-fungal agents have significant toxic side effects. Therefore, the possibility of using tetracyclines as anti-fungal agents appears attractive. Clearly, however, the state of the art teachings regarding the clinical efficacy of tetracyclines as anti-fugal agents, as described above, is contradictory.

As stated above, many patients, such as patients with compromised immune systems, are susceptible to both mucositis and fungal infections. Accordingly, there is a need for a method of simultaneously treating a patient suffering from both types of infections. It is especially advantageous if a single agent would be effective to treat both types of infections. The use of a single agent would reduce both the cost and side effects of treatment.

### SUMMARY OF THE INVENTION

The present invention provides a method for simultaneously treating mucositis and fungal infection in a mammal in need thereof. The method comprises administering to the mammal an effective amount of an anti-mucositis and anti-fungal pharmaceutical composition consisting of a tetracycline compound in an amount that is effective to simultaneously treat mucositis and fungal infection, but has substantially no antibiotic activity.

### BRIEF DESCRIPTION OF THE DRAWINGS

FIG. 1 shows the photoirritancy factor (PIF) for some tetracycline compounds. For structure K, the compounds indicated are as follows:

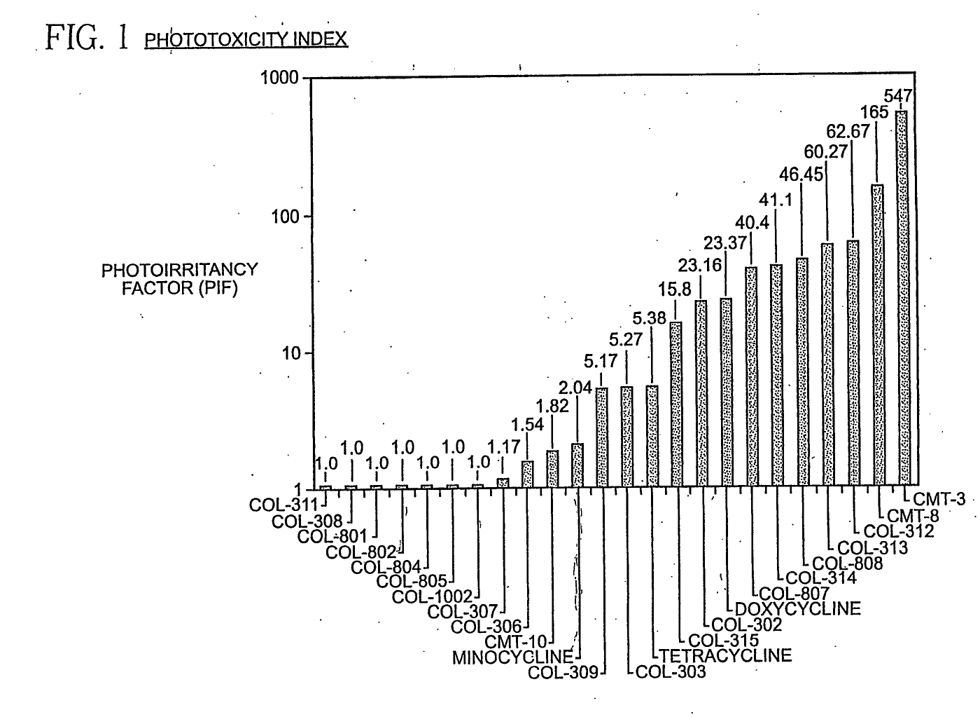

| COL | R7 | R8 | R9 |
|---|---|---|---|
| 308 | hydrogen | hydrogen | amino |
| 311 | hydrogen | hydrogen | palmitamide |
| 306 | hydrogen | hydrogen | dimethylamino |

For structures L, M, N or 0 the compounds indicated are as follows:

| COL | R7 | R8 | R9 |
|---|---|---|---|
| 801 | hydrogen | hydrogen | acetamido |
| 802 | hydrogen | hydrogen | dimethylaminoacetamido |
| 804 | hydrogen | hydrogen | nitro |
| 805 | hydrogen | hydrogen | amino |

For structure P, R8 is hydrogen and R9 is nitro (COL-1002).

FIG. 2 shows a Sample Dose Response Curve of the Positive Control Chlorpromazine for use in PIF calculations.

FIG. 3 shows a Sample Dose Response Curve for use in MPE calculations.

### DETAILED DESCRIPTION OF INVENTION

The present invention provides methods of simultaneously treating mucositis and fungal infection in a mammal.

Mucositis, as defined herein, includes any inflammation of the mucosa. The mucosa refers to the epithelial tissue that lines the internal cavities of the body. For example, the mucosa comprises the alimentary canal, including the mouth, esophagus, stomach, intestines, and anus; the respiratory tract, including the nasal passages, trachea, bronchi, and lungs; and the genitalia.

A fungal infection as defined herein includes any infection caused by fungi. Fungi include any eukaryotic single celled organism characterized by the absence of chlorophyll and by the presence of a rigid cell wall. The fungi of interest in the present specification are clinically significant fungi, i.e. fungi which grow in or on mammals. Examples of clinically significant fungi include Cryptococcus species, Canidida albicans, Rhizopus species, Aspergillus fumigatus, Penicillium species, Absidia species, Scedosporium apiospermum, Phialophora verrucosa, Cunninghamella species, Tricothecium species, Ulocladium species, and Fonsecae species.

The method of simultaneously treating mucositis and fungal infection comprises the administration of an anti-mucositis and anti-fungal pharmaceutical composition consisting of a tetracycline compound. The tetracycline compound is administered in an amount which is effective to simultaneously treat mucositis and a fungal infection, but which has substantially no antibiotic activity.

The tetracyclines are a class of compounds of which tetracycline is the parent compound. The tetracycline compounds include their pharmaceutically acceptable salts. Tetracycline has the following structure: 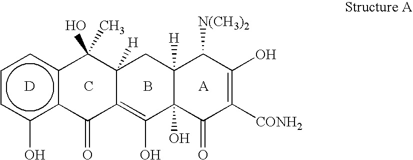
The numbering system of the multiple ring nucleus is as follows:

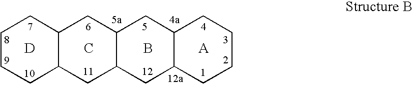

Tetracycline, as well as the 5-OH (oxytetracycline, e.g. Terramycin) and 7-Cl (chlorotetracycline, e.g. Aureomycin) derivatives, exist in nature, and are all well known antibiotic compounds. Semisynthetic derivatives such as 7-dimethylaminotetracycline (minocycline) and 6α-deoxy-5-hydroxytetracycline (doxycycline) are also known tetracycline antibiotic compounds.

Some examples of antibiotic tetracycline compounds include doxycycline, minocycline, tetracycline, oxytetracycline, chlortetracycline, demeclocycline, lymecycline, and sancycline. Doxycycline is preferably administered as its hyclate salt or as a hydrate, preferably monohydrate.

Non-antibiotic tetracycline compounds are structurally related to the antibiotic tetracyclines, but have had their antibiotic activity substantially or completely eliminated by chemical modification, as discussed in more detail below. For example, non-antibiotic tetracycline compounds are incapable of achieving antibiotic activity comparable to that of doxycline unless the concentration of the non-antibiotic tetracycline is at least about ten times, preferably at least about twenty five times, greater than that of doxycycline.

Examples of chemically modified non-antibiotic tetracyclines (CMT's) include, 4-de(dimethylamino)tetracycline (CMT-1), tetracyclinonitrile (CMT-2), 6-demethyl-6-deoxy-4-de(dimethylamino)tetracycline (CMT-3), 7-chloro-4-de(dimethylamino)tetracycline (CMT-4), tetracycline pyrazole (CMT-5), 4-hydroxy-4-de(dimethylamino)tetracycline (CMT-6), 4-de(dimethylamino)-12α-deoxytetracycline (CMT-7), 6-deoxy-5α-hydroxy-4-de(dimethylamino)tetracycline (CMT-8), 4-de(dimethylamino)-12α-deoxyanhydrotetracycline (CMT-9), 4-de(dimethylamino)minocycline (CMT-10). (COL and CMT are used interchangeably throughout this specification.)

Tetracycline derivatives, for purposes of the invention, may be any tetracycline derivative, including those compounds disclosed generically or specifically in U.S. patent application Ser. No. 09/573,653, filed on May 18, 2000; International Application No. PCT/US01/16272 filed on May 18, 2001; and U.S. patent application Ser. No. 10/274,841, filed Oct. 18, 2002, which are herein incorporated by reference. Some examples of chemically modified non-antibiotic tetracyclines include Structures C-Z. (See Index of Structures.)

The tetracycline compounds can be in the form of pharmaceutically acceptable salts of the compounds. Pharmaceutically acceptable salts may be prepared from the corresponding tetracycline compounds and an acid or base. The acids may be inorganic or organic acids. Examples of inorganic acids include hydrochloric, hydrobromic, nitric hydroiodic, sulfuric, and phosphoric acids. Examples of organic acids include carboxylic and sulfonic acids. The organic acids may be aliphatic, aromatic, aliphatic-aromatic or aromatic-aliphatic. Some examples of organic acids include formic, acetic, phenylacetic, propionic, succinic, glycolic, glucuronic, maleic, furoic, glutamic, benzoic, toluic, anthranilic, salicylic, mandelic, embonic (pamoic), methanesulfonic, ethanesulfonic, panthenoic, benzenesulfonic, stearic, sulfanilic, alginic, tartaric, citric, gluconic, gulonic, arylsulfonic, and galacturonic acids. Appropriate organic bases may be selected, for example, from N,N-dibenzylethylenediamine, chloroprocaine, choline, diethanolamine, ethylenediamine, meglumine (N-methylglucamine), and procaine.

The tetracycline compound is administered in an amount that is effective to simultaneously treat mucositis and fungal infection, but has substantially no antibiotic activity. A treatment is effective if it causes a reduction or inhibition of the symptoms associated with mucositis and fungal infection.

The minimal effective amount of the tetracycline compound administered to a mammal is the lowest amount capable of providing effective simultaneous treatment of mucositis and fungal infection. Some examples of minimal amounts include 10%, 20%, 30% and 40% of an antibiotic amount.

The maximal effective amount of the tetracycline compound administered to a mammal is the highest amount that does not significantly prevent the growth of microbes, e.g. bacteria. Some examples of maximal amounts include 50%, 60%, 70% and 80% of an antibiotic amount.

The amount of a tetracycline compound which is administered can be measured by daily dose and by serum level.

Tetracycline compounds that have significant antibiotic activity may, for example, be administered in a dose which is 10-80% of the antibiotic dose. More preferably, the antibiotic tetracycline compound is administered in a dose which is 40-70% of the antibiotic dose.

Antibiotic daily doses are known in art. Some examples of antibiotic doses of members of the tetracycline family include 50, 75, and 100 mg/day of doxycycline; 50, 75, 100, and 200 mg/day of minocycline; 250 mg of tetracycline one, two, three, or four times a day; 1000 mg/day of oxytetracycline; 600 mg/day of demeclocycline; and 600 mg/day of lymecycline.

Examples of the maximum non-antibiotic doses of tetracyclines based on steady-state pharmacokinetics are as follows: 20 mg/twice a day for doxycycline; 38 mg of minocycline one, two, three or four times a day; and 60 mg of tetracycline one, two, three or four times a day.

In a preferred embodiment, doxycycline is administered in a daily amount of from about 30 to about 60 milligrams, but maintains a concentration in human plasma below the threshold for a significant antibiotic effect.

In an especially preferred embodiment, doxycycline hyclate is administered at a 20 milligram dose twice daily. Such a formulation is sold for the treatment of periodontal disease by CollaGenex Pharmaceuticals, Inc. of Newtown, Pa. under the trademark Periostat®.

The administered amount of a tetracycline compound described by serum levels follows.

An antibiotic tetracycline compound is advantageously administered in an amount that results in a serum tetracycline concentration which is 10-80%, preferably 40-70%, of the minimum antibiotic serum concentration. The minimum antibiotic serum concentration is the lowest concentration known to exert a significant antibiotic effect.

Some examples of the approximate antibiotic serum concentrations of members of the tetracycline family follow. A single dose of two 100 mg minocycline HCl tablets administered to adult humans results in minocycline serum levels ranging from 0.74 to 4.45 μg/ml over a period of an hour. The average level is 2.24 μg/ml.

Two hundred and fifty milligrams of tetracycline HCl administered every six hours over a twenty-four hour period produces a peak plasma concentration of approximately 3 μg/ml. Five hundred milligrams of tetracycline HCl administered every six hours over a twenty-four hour period produces a serum concentration level of 4 to 5 μg/ml.

In one embodiment, the tetracycline compound can be administered in an amount which results in a serum concentration between about 0.1 and 10.0 μg/ml, more preferably between 0.3 and 5.0 μg/ml. For example, doxycycline is administered in an amount which results in a serum concentration between about 0.1 and 0.8 μg/ml, more preferably between 0.4 and 0.7 μg/ml.

Some examples of the plasma antibiotic threshold levels of tetracyclines based on steady-state pharmacokinetics are as follows: 1.0 μg/ml for doxycycline; 0.8 μg/ml for minocycline; and 0.5 μg/ml for tetracycline.

Non-antibiotic tetracycline compounds can be used in higher amounts than antibiotic tetracyclines, while avoiding the indiscriminate killing of microbes, and the risk of emergence of resistant microbes. For example, 6-demethyl-6-deoxy-4-de(dimethylamino)tetracycline (CMT-3) may be administered in doses of about 40 to about 200 mg/day, or in amounts that result in serum levels of about 1.55 μg/ml to about 10 μg/ml.

The actual preferred amounts of tetracycline compounds in a specified case will vary according to the particular compositions formulated, the mode of application, the particular sites of application, and the subject being treated (e.g. age, gender, size, tolerance to drug, etc.)

Preferably, the tetracycline compounds have low phototoxicity, or are administered in an amount that results in a serum level at which the phototoxicity is acceptable. Phototoxicity is a chemically-induced photosensitivity that occurs upon exposure to light, in particular ultraviolet light. Such photosensitivity renders skin susceptible to damage, e.g. sunburn, blisters, accelerated aging, erythemas and eczematoid lesions. The preferred amount of the tetracycline compound produces no more phototoxicity than is produced by the administration of a 40 mg total daily dose of doxycycline.

There are several methods by which to quantify phototoxicity. One method is called photoirritancy factor (PIF). The PIF is the ratio of an IC₅₀ value in the absence of light to an IC₅₀ value in the presence of light.

In calculating PIF values, the data resulting from the assay procedure can be interpreted by different methods. For example, during the period Mar. 2, 1999 to Apr. 16, 1999, PIF values were obtained using the phototoxicity software and its curve-fitting algorithms available at the time. In the present specification, this earlier phototoxicity calculation is referred to as PIF 1. At a PIF1 value of 1, a compound is considered to have no measurable phototoxicity. A PIF1 value greater than 5 is indicative of phototoxic potential of a compound.

As explained in more detail in Example 37 below, 3T3 phototoxicity assay has undergone extensive validation since April 1999, and has now been incorporated into a draft guideline by the Organization of Economic Cooperation and Development (OECD) (Draft Guideline 432). In the present specification, this revised phototoxicity calculation is referred to as PIF2. A PIF2 value of less than 2 is considered non-phototoxic, 2 to less than 5 is considered potentially phototoxic, and 5 or greater is considered clearly phototoxic.

PIF2 values are more refined than the PIF1 values. Qualitatively the differences between the PIF1 and PIF2 values are not significant. For example, the mean PIF1 values for COL 10 and COL 1002 are 1.82 and 1.0, respectively. The mean PIF2 values of COL 10 and COL 1002 are 2.04 and 1.35, respectively.

As explained in the Examples section, PIF values cannot be determined for many compounds. Another method by which to quantify relative phototoxicity is called mean photo effect (MPE). MPE values can be determined for compounds in virtually all cases. Thus, MPE values are more consistent and reliable than PFE values.

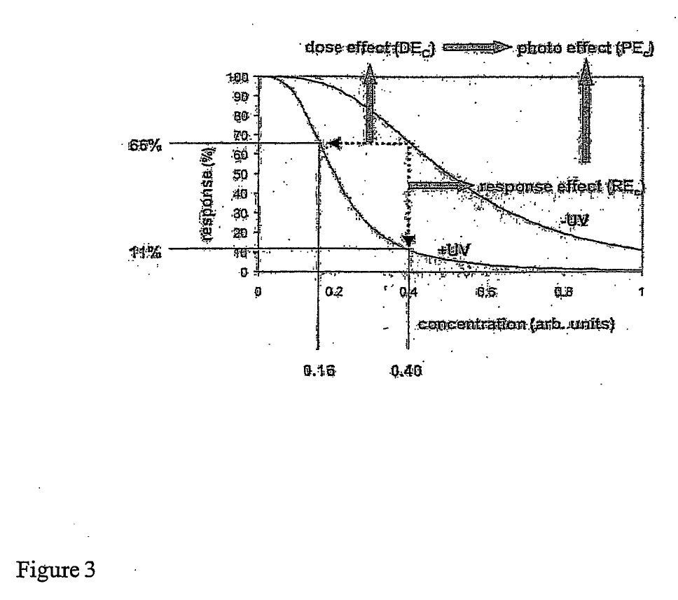

The MPE is a measure of the difference between the cytotoxicity induced by the test chemical in the presence and absence of light. It compares the responses over the range of doses selected using the two dose-response curves produced from the boot-strap analysis of the individual data points (Holzhütter 1995 and 1997). An example is provided in FIG. 3 (Peters and Holzhütter (2002)). This method of analysis is particularly suited to cases where the IC₅₀ value cannot be calculated for one or both concentration response curves.

MPE values of <0.1 (including negative values) are considered indicative of a nonphototoxin, values of 0.1 to <0.15 are considered probable phototoxins, and values greater than and equal to 0.15 are considered to be clear phototoxins.

A class of low phototoxicity tetracyline derivatives has less than approximately 75% of the phototoxicity of minocycline, preferably less than approximately 70%, more preferably less than approximately 60%, and most preferably less than approximately 50%. Minocycline has a PIF1 of about 2.04, and an MPE of about 0.041.

The class of low phototoxicity tetracycline compound derivatives includes those derivatives having PIF 1 or PIF 2 values of approximately 1, i.e. 1 to about 2, preferably 1 to about 1.5. The class of low phototoxicity tetracycline derivatives optimally have MPE values of less than 0.1. Members of this class include, but are not limited to, tetracycline compounds having general formulae:

### Structure K

wherein: R7, R8, and R9 taken together in each case, have the following meanings:

| R7 | R8 | R9 |
|---|---|---|
| hydrogen | hydrogen | amino |
| hydrogen | hydrogen | palmitamide |
| hydrogen | hydrogen | dimethylamino |
| trimethylammonium | hydrogen | hydrogen |
| and | | |

| | |
|---|---|
| STRUCTURE L | STRUCTURE M |
| STRUCTURE N | STRUCTURE O |

wherein: R7, R8, and R9 taken together in each case, have the following meanings:

| R7 | R8 | R9 |
|---|---|---|
| hydrogen | hydrogen | acetamido |
| hydrogen | hydrogen | dimethylaminoacetamido |
| hydrogen | hydrogen | nitro |
| hydrogen | hydrogen | amino |
| and | | |

STRUCTURE P

wherein: R8 and R9 taken together are, respectively, hydrogen and nitro.

The tetracycline compounds are preferably administered systemically or topically. For the purposes of this specification, “systemic administration” means administration to a human by a method that causes the compounds to be absorbed into the bloodstream.

For example, the tetracycline compounds can be administered orally by any method known in the art. For example, oral administration can be by tablets, capsules, pills, troches, elixirs, suspensions, syrups, wafers, chewing gum and the like.

Additionally, the tetracycline compounds can be administered enterally or parenterally, e.g., intravenously, intramuscularly, or subcutaneously, as injectable solutions or suspensions; intraperitoneally; or rectally. Administration can also be intranasally, in the form of, for example, an intranasal spray; or transdermally, in the form of, for example, a patch.

For the pharmaceutical purposes described above, the tetracycline compounds 5 can be formulated in pharmaceutical preparations optionally with a suitable pharmaceutical carrier (vehicle) or excipient as understood by practitioners in the art. These preparations can be made according to conventional chemical methods.

In the case of tablets and capsules for oral use, carriers which are commonly used include lactose and corn starch. Lubricating agents such as magnesium stearate are commonly added. Further examples of carriers and excipients include milk, sugar, certain types of clay, gelatin, stearic acid or salts thereof, calcium stearate, talc, vegetable fats or oils, gums and glycols.

When aqueous suspensions are used for oral administration, emulsifying and/or suspending agents are commonly added. In addition, sweetening and/or flavoring agents may be added to the oral compositions.

For intramuscular, intraperitoneal, subcutaneous and intravenous use, sterile solutions of the tetracycline compounds can be employed. The pH of the solutions are preferably adjusted and buffered. For intravenous use, the total concentration of the solute(s) can be controlled in order to render the preparation isotonic.

The tetracycline compounds of the present invention optionally further comprise one or more additional pharmaceutically acceptable ingredient(s) such as alum, stabilizers, buffers, coloring agents, flavoring agents, and the like.

The tetracycline compound may be administered intermittently. For example, the tetracycline compound may be administered 1-6 times a day, preferably 1-4 times a day.

Alternatively, the tetracycline compound may be administered by sustained release. Sustained release administration is a method of drug delivery to achieve a certain level of the drug over a particular period of time. The level typically is measured by serum concentration. Further description of methods of delivering tetracycline compounds by sustained release can be found in the patent application, “Controlled Delivery of Tetracycline and Tetracycline Derivatives,” filed on Apr. 5, 2001 and assigned to CollaGenex Pharmaceuticals, Inc. of Newtown, Pa. The aforementioned application is incorporated herein by reference in its entirety. For example, 40 milligrams of doxycycline may be administered by sustained release over a 24 hour period.

For topical application, the tetracycline compounds are placed in carrier compositions deemed to be suited for topical use, such as gels, salves, lotions, creams, ointments and the like. The carrier compositions can also be incorporated into a support base or matrix which can be directly applied to the mucosa. Examples of a support base or matrix include gauze or bandages.

The carrier compositions can comprise a tetracycline compound in amounts of up to about 25% (w/w). Amounts of from about 0.1% to about 10% are preferred.

Topical application is preferred for particular non-antibiotic tetracycline compounds which have only limited biodistribution, e.g. CMT-5.

Combined or coordinated topical and systemic administration of the tetracycline compounds is also contemplated under the invention. For example, a systemically non-absorbable non-antibiotic tetracycline compound can be administered topically, while a tetracycline compound capable of substantial absorption and effective systemic distribution in a human can be administered systemically.

The tetracycline compounds are prepared by methods known in the art. For example, natural tetracyclines may be modified without losing their antibiotic properties, although certain elements of the structure must be retained. The modifications that may and may not be made to the basic tetracycline structure have been reviewed by Mitscher in The Chemistry of Tetracyclines, Chapter 6, Marcel Dekker, Publishers, New York (1978). According to Mitscher, the substituents at positions 5-9 of the tetracycline ring system may be modified without the complete loss of antibiotic properties. Changes to the basic ring system or replacement of the substituents at positions 1-4 and 10-12, however, generally lead to synthetic tetracyclines with substantially less or effectively no antibiotic activity.

Further methods of preparing the tetracycline compounds are described in the examples.

### EXAMPLES

The following examples serve to provide further appreciation of the invention but are not meant in any way to restrict the effective scope of the invention.

Preparation of Compounds

### Example 1

### 4-Dedimethylamino-7-dimethylamino-6-demethyl-6-deoxy-9-nitrotetracycline sulfate

To a solution of one millimole of 4-dedimethylamino-7-dimethylamino-6-demethyl-6-deoxytetracycline in 25 ml of concentrated sulfuric acid at 0° C. was added 1.05 mmole of potassium nitrate. The resulting solution was stirred at ice bath temperature for 15 minutes and poured in one liter of cold ether with stirring. The precipitated solid was allowed to settle and the majority of solvent decanted. The remaining material was filtered through a sintered glass funnel and the collected solid was washed well with cold ether. The product was dried in a vacuum desiccator overnight.

### Example 2

### 9-amino-dedimethylamino-7diethylaamino-6-demethyl-6-deoxytetracycline sulfate

To a solution of 300 mg of the 9-nitro compound from example 1, in 30 ml of ethanol was added 50 mg of Pt0₂. The mixture was hydrogenated at atmospheric pressure until the theoretical amount of hydrogen was absorbed. The system is flushed with nitrogen, the catalyst PtO₂ is filtered and the filtrate added dropwise to 300 ml of ether. The product that separates is filtered and dried in a vacuum desiccator.

### Example 3

### 9-Acetamido-4-dedimethylamino-7-dimethylamino-6-demethyl-6-deoxytetracycline sulfate

To a well stirred cold solution of 500 mg of 9-amino-4-dedimethylamino-7-dimethylamino-6-demethyl-6-deoxytetracycline sulfate from example 2, in 2.0 ml of 1.3-dimethyl-2-imidizolidinone, 500 mg of sodium bicarbonate was added followed by 0.21 ml of acetyl chloride. The mixture is stirred at room temperature for 30 minutes, filtered and the filtrate was added dropwise to 500 ml of ether. The product that separated was filtered and dried in a vacuum desiccator.

### Example 4

### 4-Dedimethylamino-7-dimethylamino-6-demethyl-6-deoxy-9-diazoniumtetracycline sulfate

To a solution of 0.5 g of 9-amino-4-dedimethylamino-7-dimethylamino-6-demethyl-6-deoxytetracycline sulfate, from example 2, in 10 ml of 0.1N hydrochloric acid in methanol cooled in an ice bath, 0.5 ml of n-butyl nitrite was added. The solution was stirred at ice bath temperature for 30 minutes and then poured into 250 ml of ether. The product that separated was filtered, washed with ether and dried in a vacuum desiccator.

### Example 5

### 9-Azido-4-dedimethylamino-7-dimethylamino-6-demethyl-6-deoxytetracycline sulfate

To a solution of 0.3 mmole of 4-dedimethylamino-7-dimethylamino-6-demethyl-6-deoxy-9-diazoniumtetracycline sulfate, from example 4, 10 ml of 0.1 N methanolic hydrogen chloride was added 0.33 mmole of sodium azide. The mixture was stirred at room temperature for 1.5 hours. The reaction mixture was then poured into 200 ml of ether. The product that separated was filtered and dried in a vacuum desiccator.

### Example 6

### 9-Amino-8-chloro-4-dedimethylamino-7-dimethylamino-6-demethyl-6-deoxy-tetracycline sulfate

One gram of 9-azido-4-dedimethylamino-7-dimethylamino-6-demethyl-6-deoxytetracycline hydrochloride, from example 4, was dissolved in 10 ml of concentrated sulfuric acid saturated with HCL at 0° C. The mixture was stirred at ice bath temperature for 1.5 hours and then slowly added dropwise to 500 ml of cold ether. The product that separated was filtered, washed with ether and dried in a vacuum desiccator.

### Example 7

### 4-Dedimethylamino-7-dimethylamino-6-demethyl-6-deoxy-9-ethoxythiocarbonylthio-tetracycline sulfate

A solution of 1.0 mmole of 4-dedimethylamino-7-dimethylamino-6-demethyl-6-deoxy-9-diazoniumtetracycline sulfate, from example 4, in 15 ml of water was added to a solution of 1.15 mmole of potassium ethyl xanthate in 15 ml of water. The mixture was stirred at room temperature for one hour. The product separated and was filtered and dried in a vacuum desiccator.

### Example 8A

### General Procedure for Nitration

To 1 mmole of a 4-dedimethylamino-6-deoxytetracycline in 25 ml of concentrated sulfuric acid at 0° C. was added 1 mmole of potassium nitrate with stiring. The reaction solution was stirred for 15 minutes and then poured into 100 g of chopped ice. The aqueous solution was extracted 5 times with 20 ml of butanol each time. The butanol extracts were washed three times with 10 ml of water each time, and concentrated in vacuo to a volume of 25 ml. The light yellow crystalline solid which precipitated was filtered, washed with 2 ml of butanol and dried in vacuo at 60° C. for 2 hours. This solid was a mixture of the two mononitro isomers.

### Example 8B

### 4-Dedimethylamino-6-deoxy-9-nitrotetracycline

To 980 mg of the nitration product from 4-dedimethylamino-6-deoxytetracycline (a mixture of the 2 isomers) in 25 ml of methanol was added enough triethylamine to dissolve the solid. The filtered solution (pH 9.0) was adjusted to pH 5.2 with concentrated sulfuric acid. A crystalline yellow solid (236 mg.) was obtained (29% yield). The material at this point was quite pure and contained only small amounts of the 7-isomer. Final purification was accomplished by liquid partition chromatography using a diatomaceous earth packed column and the solvent system: chloroform: butanol: 0.5 M phosphate buffer (pH 2) (16:1:10).

### Example 9

### 4-Dedimethylamino-6-deoxy-7-nitrotetracycline

The methanol filtrate from example 8 was immediately adjusted to pH 1.0 with concentrated sulfuric acid. The light yellow crystalline solid, which was obtained as the sulfate salt. A purified free base was obtained by adjusting an aqueous solution of the sulfate salt (25 mg/ml) to pH 5.2 with 2 N sodium carbonate.

### Example 10

### 9-Amino-4-dedimethylamino-6-deoxytetracycline

To a solution of 300 mg of the 9-nitro compound, prepared in example 8, in 30 ml of ethanol was added 50 mg of PtO₂. The mixture was hydrogenated at atmospheric pressure until the theoretical amount of hydrogen was absorbed. The system is flushed with nitrogen, the PtO2 catalyst is filtered and the filtrate added dropwise to 300 ml of ether. The solid that separates is filtered and dried in a vacuum desiccator.

### Example 11

### 9-Acetamido-4-dedimethylamino-6-deoxytetracycline sulfate

To well stirred cold solution of 500 mg of 9-amino4-dedimethylamino-6-deoxytetracycline sulfate, from example 10, in 2.0 ml of 1,3-dimethyl-2-imidazolidinone was added 500 mg of sodium bicarbonate followed by 0.21 ml of acetyl chloride. The mixture was stirred at room temperature for 30 minutes, filtered and the filtrate was added dropwise to 500 ml of ether. The solid that separated was filtered and dried in a vacuum desiccator.

### Example 12

### 4-Dedimethylamino-6-deoxy-9-diazoniumtetracycline sulfate

To a solution of 0.5 g of 9-amino4-dedimethylamino-6-deoxytetracycline sulfate, from example 10, in 10 ml of 0.1N hydrochloric acid in methanol cooled in an ice bath was added 0.5 ml of n-butyl nitrite. The solution was stirred at ice bath temperature for 30 minutes and the poured into 250 ml of ether. The solid that separated was filtered, washed with ether and dried in a vacuum desiccator.

### Example 13

### 9-Azido4-dedimethylamino-6-deoxytetracycline sulfate

To a solution of 0.3 mmole of 4-dedimethylamino-6-deoxy-9-diazoniumtetracycline sulfate, of example 12, 10 ml of 0.1 N methanolic hydrogen chloride was added 0.33 mmole of sodium azide. The mixture was stirred at room temperature for 1.5 hours. The reaction mixture was then poured into 200 ml of ether. The solid that separated was filtered and dried in a vacuum desiccator.

### Example 14

### 9-Amino-8-chloro4-dedimethylamino-6-deoxytetracycline sulfate

One gram of 9-azido-4-dedimethylamino-7-dimethylamino-6-deoxytetracycline hydrochloride, from example 13, was dissolved in 10 ml of concentrated sulfuric acid saturated with HCL at 0° C. The mixture was stirred at ice bath temperature for 1.5 hours and then slowly added dropwise to 500 ml of cold ether. The solid that separated was filtered, washed and ether and dried in a vacuum desiccator.

### Example 15

### 4-Dedimethylamino-6-deoxy-9-ethoxythiocarbonylthiotetracycline sulfate

A solution of 1.0 mmole of 4-dedimethylamino-6-deoxy-9-diazoniumtetracycline sulfate, from example 12, in 15 ml of water was added to a solution of 1.15 mmole of potassium ethyl xanthate in 15 ml of water. The mixture was stirred at room temperature for one hour. The solid that separated was filtered and dried in a vacuum desiccator.

### Example 16

### 9-Dimethylamino-4-dedimethylamino-6-deoxytetracycline sulfate

To a solution of 100 mg. of the 9-amino compound from example 10, in 10 ml of ethylene glycol monomethyl ether is added 0.05 ml of concentrated sulfuric acid, 0.4 ml. of a 40% aqueous formaldehyde solution and 100 mg of a 10% palladium on carbon catalyst. The mixture is hydrogenated under atmospheric pressure and room temperature for 20 minutes. The catalyst was filtered and the filtrate was evaporated to dryness under reduced pressure. The residue is dissolved in 5 ml of methanol and this solution was added to 100 ml of ether. The product that separated was filtered and dried, yield, 98 mg.

### Example 17

### 7-Amino-4-dedimethylamino-6-deoxytetracycline

This compound can be made using Procedure A or B. Procedure A. To a solution of 300 mg of the 7-nitro compound, from example 1, in 30 ml of ethanol was added 50 mg of PtO₂. The mixture was hydrogenated at atmospheric pressure until the theoretical amount of hydrogen was absorbed. The system is flushed with nitrogen, the catalyst PtO₂ is filtered and the filtrate added dropwise to 300 ml of ether. The solid that separates is filtered and dried in a vacuum desiccator.

Procedure B. 1 g of 6-deoxy4-dedimethylamino-tetracycline was dissolved in 7.6 ml THF and 10.4 ml methanesulfonic acid at −10° C. After warming the mixture to 0° C. a solution of 0.86 g of dibenzyl azodicarboxylate was added and the mixture stirred for 2 hours at 0° C. to yield 7-[1,2-bis(carbobenzyloxy)hydrazino]-4-dedimethylamino-6-deoxytetracycline. A solution of 1 millimole of this material in 70 ml 2-methoxyethanol, and 300 mg 10% Pd-C was hydrogenated at room temperature to give 7-amino-6-deoxy-4-dedimethylaminotetracycline.

### Example 18

### 7-Amino-6-deoxy-5-hydroxy-4-dedimethylaminotetracycline

1 g of 6-deoxy-5-hydroxy-4-dedimethylaminotetracycline 3 was dissolved in 7.6 ml THF and 10.4 ml methanesulfonic acid at −10° C. After warming the mixture to 0° C. a solution of 0.86 g dibenzyl azodicarboxylate in 0.5 ml THF was added and the mixture stirred for 2 hours at 0° C. to yield 7-[1,2-bis(carbobenzyloxy)hydrazino]-4-dedimethylamino-6-deoxy-5-hydroxytetracycline. A solution of 1 millimole of this material in 70 ml 2-methoxyethanol, and 300 mg 10% Pd-C was hydrogenated at room temperature to give 7-amino-6-deoxy-5-hydroxytetracycline.

### Example 19

### 7-Acetamido-4-dedimethylamino-6-deoxy-5-hydroxytetracycline sulfate.

To well stirred cold solution of 500 mg of 7-amino-4-dedimethylamino-6-deoxy-5-hydroxytetracycline sulfate, from example 18, in 2.0 ml of 1,3-dimethyl-2-imidazolidinone was added 500 mg of sodium bicarbonate followed by 0.21 ml of acetyl chloride. The mixture was stirred at room temperature for 30 minutes, filtered and the filtrate was added dropwise to 500 ml of ether. The solid that separated was filtered and dried in a vacuum desiccator.

### Example 20

### 4-Dedimethylamino-6-deoxy-5-hydroxy-7-diazoniumtetracycline hydrochloride

To a solution of 0.5 g of 7-amino-4-dedimethylamino-6-deoxy-5-hydroxytetracycline sulfate, from example 20, in 10 ml of 0.1N hydrochloric acid in methanol cooled in an ice bath was added 0.5 ml of n-butyl nitrite. The solution was stirred at ice bath temperature for 30 minutes and then poured into 250 ml of ether. The solid that separated was filtered, washed with ether and dried in a vacuum desiccator.

### Example 21

### 7-Azido-4-Dedimethylamino-6-deoxy-5-hydroxytetracycline

To a solution of 0.3 mmole of 4-dedimethylamino-6-deoxy-5-hydroxy-7-diazoniumtetracycline hydrochloride, from example 20, 10 ml of 0.1 N methanolic hydrogen chloride was added 0.33 mmole of sodium azide. The mixture was stirred at room temperature for 1.5 hours. The reaction mixture was then poured into 200 ml of ether. The solid that separated was filtered and dried in a vacuum desiccator.

### Example 22

### 7-Amino-8-chloro-4-dedimethylamino-6-deoxy-5-hydroxytetracycline sulfate

One gram of 7-azido-4-dedimethylamino-7-dimethylamino-6-deoxy-5-hydroxytetracycline sulfate, from example 21, was dissolved in 10 ml of concentrated sulfuric acid (previously saturated with hydrogen chloride) at 0° C. The mixture was stirred at ice bath temperature for 1.5 hours and then slowly added dropwise to 500 ml of cold ether. The solid that separated was filtered, washed with ether and dried in a vacuum desiccator.

### Example 23

### 4-Dedimethylamino-6-deoxy-5-hydroxy-7-ethoxythiocarbonylthiotetracycline

A solution of 1.0 mmole of 4-dedimethylamino-6-deoxy-5-hydroxy-7-diazoniumtetracycline hydrochloride, from example 20, in 15 ml of water was added to a solution of 1.15 mmole of potassium ethyl xanthate in 15 ml of water. The mixture was stirred at room temperature for one hour. The solid that separated was filtered and dried in a vacuum desiccator.

### Example 24

### 7-Dimethylamino-4-dedimethylamino-6-deoxy-5-hydroxytetracycline sulfate

To a solution of 100 mg of the 7-amino compound in 10 ml of ethylene glycol monomethyl ether is added 0.05 ml of concentrated sulfuric acid, 0.4 ml of a 40% aqueous formaldehyde solution and 100 mg of a 10% palladium on carbon catalyst. The mixture is reduced with hydrogen at atmospheric pressure and room temperature for 20 minutes. The catalyst was filtered and the filtrate was evaporated to dryness under reduced pressure. The residue is dissolved in 5 ml of methanol and this solution was added to 100 ml of ether. The product that separated was filtered and dried, yield, 78 mg.

### Example 25

### 7-Diethylamino-4-dedimethylamino-5-hydroxytetracycline sulfate

To a solution of 100 mg of the 7-amino compound in 10 ml of ethylene glycol monomethyl ether is added 0.05 ml of concentrated sulfuric acid, 0.4 ml of acetaldehyde and 100 mg of a 10% palladium on carbon catalyst. The mixture is reduced with hydrogen at atmospheric pressure at room temperature for 20 minutes. The catalyst was filtered and filtrate was evaporated to dryness under reduced pressure. The residue is dissolved in 5 ml of methanol and this solution was added to 100 ml of ether. The product that separated was filtered and dried.

### Example 26

### 4-Dedimethylamino-6-deoxy-7-diazoniumtetracycline hydrochloride

To a solution of 0.5 g. of 7-amino-4-dedimethylamino-6-deoxytetracycline sulfate, from example 17, in 10 ml of 0.1N hydrochloric acid in methanol cooled in an ice bath was added 0.5 ml of n-butyl nitrite. The solution was stirred at ice bath temperature for 30 minutes and then poured into 250 ml of ether. The solid that separated was filtered, washed with ether and dried in a vacuum desiccator.

### Example 27

### 7-Azido-4-dedimethylamino-6-deoxytetracycline

To a solution of 0.3 mmole of 4-dedimethylamino-6-deoxy-7-diazoniumtetracycline hydrochloride, from example 26, 10 ml of 0.1 N methanolic hydrogen chloride was added 0.33 mmole of sodium azide. The mixture was stirred at room temperature for 1.5 hours. The reaction mixture was then poured into 200 ml of ether. The solid that separated was filtered and dried in a vacuum desiccator.

### Example 28

### 7-Amino-8-chloro-4-dedimethylamino-6-deoxytetracycline sulfate

One gram of 7-azido-4-dedimethylamino-7-dimethylamino-6-deoxytetracycline sulfate was dissolved in 10 ml of concentrated sulfuric acid (previously saturated with hydrogen chloride) at 0° C. The mixture was stirred at ice bath temperature for 1.5 hours and then slowly added dropwise to 500 ml of cold ether. The solid that separated was filtered, washed with ether and dried in a vacuum desiccator.

### Example 29

### 4-Dedimethylamino-6-deoxy-7-ethoxythiocarbonylthiotetracycline

A solution of 1.0 mmole of 4-dedimethylamino-6-deoxy-7-diazoniumtetracycline hydrochloride, from example 26, in 15 ml of water was added to a solution of 1.15 mmole of potassium ethyl xanthate in 15 ml of water. The mixture was stirred at room temperature for one hour. The solid that separated was filtered and dried in a vacuum desiccator.

### Example 30

### 7-Dimethylamino-4-dedimethylamino-6-deoxytetracycline sulfate

To a solution of 100 mg of the 7-amino compound, from example 26, in 10 ml of ethylene glycol monomethyl ether is added 0.05 ml of concentrated sulfuric acid, 0.4 ml of a 40% aqueous formaldehyde solution and 100 mg of a 10% palladium on carbon catalyst. The mixture is reduced with hydrogen at atmospheric pressure and room temperature for 20 minutes. The catalyst was filtered and the filtrate was evaporated to dryness under reduced pressure. The residue is dissolved in 5 ml of methanol and this solution was added to 100 ml of ether. The product that separated was filtered and dried.

### Example 31

### 9-Acetamido-8-chloro-4-dedimethylamino-7-dimethylamino-6-deoxy-6-demethyltetracycline

To well stirred cold solution of 500 mg of 9-amino-8-chloro-4-dedimethylamino-6-deoxy-6-demethyl-7-dimethyl amino tetracycline sulfate, from example 6, in 2.0 ml of 1,3-dimethyl -2-imidazolidinone was added 500 mg of sodium bicarbonate followed by 0.21 ml. of acetyl chloride. The mixture was stirred at room temperature for 30 minutes, filtered and the filtrate was added dropwise to 500 ml of ether. The solid that separated was filtered and dried in a vacuum desiccator.

### Example 32

### 8-Chloro4-dedimethylamino-7-dimethylamino-6-deoxy-6-demethyl-9-ethoxythiocarbonylthiotetracycline

A solution of 1.0 mmole of -8-chloro4-dedimethylamino-6-deoxy-6-demethyl-7-dimethyl amino-9-diazoniumtetracycline hydrochloride in 15 ml of water was added to a solution of 1.15 mmole of potassium ethyl xanthate in 15 ml of water. The mixture was stirred at room temperature for one hour. The solid that separated was filtered and dried in a vacuum desiccator.

### Example 33

### 8-Chloro-9-dimethylamino-4-dedimethylamino-7-dimethylamino-6-deoxy-6-demethytetracycline sulfate

To a solution of 100 mg. of the 9-amino compound, from example 6, in 10 ml of ethylene glycol monomethyl ether is added 0.05 ml of concentrated sulfuric acid, 0.4 ml of acetaldehyde and 100 mg of a 10% palladium on carbon catalyst. The mixture is reduced with hydrogen at atmospheric pressure and room temperature for 20 minutes. The catalyst was filtered and the filtrate was evaporated to dryness under reduced pressure. The residue is dissolved in 5 ml of methanol and this solution was added to 100 ml of ether. The product that separated was filtered and dried.

### Example 34

### N-(4-methylpiperazin-1-yl)methyl-4-dedimethylamino-6-demethyl-6-deoxytetracycline

An aqueous solution of 58 mg (37%) formaldehyde (0.72 mmol) was added to a solution of 203 mg (0.49 mmol) of 4-dedimethylamino-6-demethyl-6-deoxytetracycline in 5.0 ml ethylene glycol dimethyl ether. The mixture was stirred at room temperature for 0.5 hours. 56 mg (0.56 mmol) of 1-methylpiperazine was then added and the resulting mixture was stirred overnight and refluxed for 20 minutes. The mixture was then cooled and a solid product was collected by filtration. The solid product was then washed with the solvent and dried by vacuum filtration.

### Example 35

### N-(4-methylpiperazin-1-yl)methyl4-dedimethylamino-6-demethyl-6-deoxy-9-hexanoylaminotetracycline

An aqueous solution of 49 mg 37 % formaldehyde (0.60 mmol) was added to a solution of 146 mg (0.30 mmol) of 4-dedimethylamino-6-demethyl-6-deoxy-9-hexanoylaminotetracycline in 5.0 ml ethylene glycol dimethyl ether. The mixture was stirred at room temperature for 0.5 hours. 60 mg (0.60 mmol) of 1-methylpiperazine was then added and the resulting mixture was stirred overnight and refluxed for 20 minutes. The mixture was then cooled and a solid product was collected by filtration. The solid product was then washed with the solvent and dried by vacuum filtration.

### Example 36

### 4-Dedimethylamino-6-demethyl-6-deoxy-9-hexanoylaminotetracycline.

1.54 g (7.2 mmol) of hexanoic anhydride and 150 mg of 10% Pd/C catalyst were added to 300 mg (0.72 mmol) of 4-dedimethylamino-6-demethyl-6-deoxytetracycline in 6.0 ml of 1,4-dioxane and 6.0 ml of methanol. The mixture was hydrogenated overnight at room temperature. The catalyst was removed by filtration and the filtrate was concentrated under reduced pressure. The residue was dissolved in 7 ml of ethyl acetate and trituated with 50 ml of hexane to produce a solid product. The solid product was filtered and dried by vacuum filtration.

### Example 37

### Phototoxicity Determination

BALB/c 3T3 (CCL-163) cells were obtained from ATCC and cultured in antibiotic-free Dulbecco's Minimum Essential Medium (4.5 g/1 glucose)(DMEM) supplemented with L-glutamine (4 mM and 10% newborn calf serum. The working cell bank was prepared and found to be free of mycoplasma. Streptomycin sulfate (100 g/ml) and penicillin (100 IU/ml) were added to the medium after the cells were treated with test article in 96-well plates.

Serial dilutions of the tetracycline derivatives were prepared in DMSO at concentrations 100× to final testing concentration. The COL dilutions in DMSO were then diluted in Hanks' Balanced Salt Solution (HBSS) for application to the cells. The final DMSO concentration was 1% in treated and control cultures. A dose range finding assay is conducted with eight serial dilutions covering a range of 100-0.03 μg/ml in half log steps. Definitive assays are conducted with 6-8 serial dilutions prepared in quarter log steps, centered on the expected 50% toxicity point as determined in the dose range finding assay. One hundred 100 μg/ml was the highest dose recommended to prevent false negative results from UV absorption by the dosing solutions. One dose range finding and at least two definitive trials were performed on each tetracycline derivative and control compound.

Controls: Each assay included both negative (solvent) and positive controls. Twelve wells of negative control cultures were used on each 96-well plate. Chlorpromazine (Sigma Chemicals) was used as the positive control and was prepared and dosed like the test tetracycline derivatives.

Solar Simulator: A Dermalight SOL 3 solar simulator, equipped with a UVA H1 filter (320-400 nm), was adjusted to the appropriate height. Measurement of energy through the lid of a 96-well microtiter plate was carried out using a calibrated UV radiometer UVA sensor. Simulator height was adjusted to deliver 1.7±0.1 mW/cm² of UVA energy (resulting dose was 1 J/cm² per 10 minutes of exposure).

Phototoxicity Assay: Duplicate plates were prepared for each test material by seeding 10⁴ 3T3 cells per well in complete medium 24 hours before treatment. Prior to treatment, the medium was removed, and the cells washed once with 125 μl of prewarmed HBSS. Fifty μl of prewarmed HBSS were added to each well. Fifty μl of each test article dilution were added to the appropriate wells and the plates returned to the incubator for approximately one hour. Six wells were treated with each dose of test or control article on each plate. Following the 1 hr incubation, the plates designated for the photo irradiation were exposed (with the lid on) to 1.7±0.1 mW/cm² UVA light for 50±2 minutes at room temperature resulting in an irradiation dose of 5 J/cm². Duplicate plates, designated for the measurement of cytotoxicity without light, were kept in the dark room temperature for 50±2 minutes. After the 50 minute exposure period (with or without light) the test article dilutions were decanted from the plates and the cells washed once with 125 μl of HBSS. One hundred μl of medium were added to all wells and the cells incubated as above for 24±1 hours.

After 24 hours of incubation, the medium was decanted and 100 μl of the Neutral Red containing medium were added to each well. The plates were returned to the incubator and incubated for approximately 3 hours. After 3 hours, the medium was decanted and each well rinsed once with 250 μl of HBSS. The plates were blotted to remove the HBSS and 100 μl of Neutral Red Solvent were added to each well. After a minimum of 20 minutes of incubation at room temperature (with shaking), the absorbance at 550 nm was measured with a plate reader, using the mean of the blank outer wells as the reference. Relative survival was obtained by comparing the amount of neutral red taken by each well treated with the test article and positive control to the neutral red taken up by the average of the negative wells (12 wells) on the same plate. The amount of neutral red taken up by the negative control wells is considered to be 100% survival.

There are several methods by which to quantify relative phototoxicity, e.g., the photoirritancy factor (PIF) and the mean photo effect (MPE), as discussed below.

Phototoxicity Determined by PIF Valuations

To determine the dose where there is a 50% decrease in relative viability, the relative cell viability is plotted as a function of increasing dose and a polynomial equation is calculated to produce the “best fit” line through all the points. The dose of a test substance corresponding to the point where this line crosses the 50% survival point is calculated (termed the Inhibitory Concentration 50% or IC₅₀) and used to compare the toxicity of the test chemical in the presence and absence of UVA/visible light.

Phototoxicity of a tetracycline derivative can be measured by its photoirritancy factor (PIF). The photo irritancy factor (PIF) is the ratio of the IC₅₀, value in the absence of light to the IC₅₀ value in the presence of light. That is, the PIF was determined by comparing the IC₅₀ without UVA [IC₅₀(−UVA)] with the IC₅₀ with UVA [IC₅₀ (+UVA)]:

$$PIF = \frac{{IC}_{50} ( - UVA )}{{IC}_{50} ( + UVA )}$$

IC₅₀ values for both the UVA exposed and non-exposed groups were determined whenever possible. If the two values are the same, the PIF is 1 and there is no phototoxic effect. If the action of the light increases toxicity, the IC₅₀ with light will be lower than the IC₅₀ without light, and the PIF will increase.

If IC₅₀ (+UVA) can be determined but IC₅₀(−UVA) cannot, the PIF cannot be calculated, although the compound tested may have some level of phototoxic potential. In this case, a “>PIF” can be calculated and the highest testable dose (−UVA) will be used for calculation of the “>PIF.”

$$> PIF = \frac{maximum dose ( - UVA )}{{IC}_{50} ( + UVA )}$$

If both, IC₅₀ (−UVA) and IC₅₀ (+UVA) cannot be calculated because the chemical does not show cytotoxicty (50% reduction in viability) up to the highest dose tested, this would indicate a lack of phototoxic potential.

In calculating PIF values, the data resulting from the assay procedure can be interpreted by different methods.

For example, during the period Mar. 2, 1999 to Apr. 16, 1999, PIF values were obtained using the earlier phototoxicity software and its curve-fitting algorithms, i.e. PIF1.

Since April 1999, the 3T3 phototoxicity assay has undergone extensive validation, and has now been incorporated into a draft guideline by the Organization of Economic Cooperation and Development (OECD) (Draft Guideline 432). (See Spielmann et al., The International EU/COLIPA In Vitro Phototoxicity Validation Study; Results of Phase II (blind trial). Part 1: The 3T3 NRU Phototoxicity Test. Toxicology In Vitro 12:305-327 (1998); and Spielmann et al., A Study on UV Filter Chemicals from Annex VII of European Union Directive 76/768/EEC, in the In Vitro 3T3 Phototoxicity Test. ATLA 26:679-708 (1998).) The new guideline follows the same assay procedure, but provides some additional guidance in the interpretation of the resulting data, and incorporates updated software. As used herein, the PIF value interpreted by this method is termed PIF2.

According to this updated OECD draft guideline, the IC₅₀ values are developed from curves fitted to the data by a multiple boot strap algorithm. The curve fitting and calculations of the PIF are performed by software developed under contract to the German government (ZEBET, Berlin).

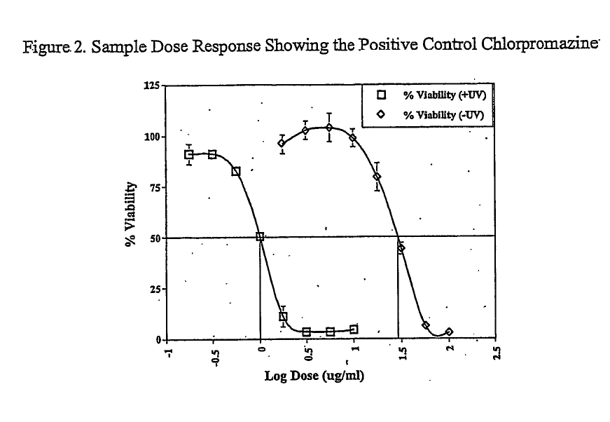

In particular, since there are six wells (and therefore six relative survival values) for each dose, the software performs multiple calculations of the best fit line using what is called boot strapping. This approach is used to account for variations in the data. From the bootstrapped curves, the software determines a mean IC₅₀ for the treatment. The IC₅₀ is used to compare the toxicity of the test chemical in the presence and absence of UVA/visible light. FIG. 2 shows an example of a set of dose response curves prepared for the positive control chemical Chlorpromazine. The difference in the IC₅₀ values can be clearly seen in this example of a highly phototoxic chemical.

Using the original software and evaluation procedures, if both IC₅₀ values can be determined, the cut off value of the factor to discriminate between phototoxicants and non-phototoxicants is a factor of 5. A factor greater than 5 is indicative of phototoxic potential of the test material. Using this software, the mean PIF1 for COL 10 was determined to be 1.83. The mean PIF1 for COL 1002 was determined to be 1.12.

The OECD draft guideline has revised the values for the PIF used to differentiate between phototoxins, potential phototoxins and non-phototoxins. A PIF2 of less than 2 is considered non-phototoxic, 2 to less than 5 is considered potentially phototoxic, and 5 or greater is considered clearly phototoxic. In accordance with the OECD draft guideline, the mean PIF2 values of COL 10 and COL 1002 are 2.04 and 1.35, respectively.

Phototoxicity Determined by MPE Valuations

At each data point, a photo effect is calculated according to the following formula:
Photo Effect_{c}=Dose Effect_{c}×Response Effect_{c} (i.e., PE_{c}=DE_{c}×RE_{c})
where c represents one concentration

Dose Effect_{c} compares the dose required to achieve percent survival n without UVA (c) with the dose required to achieve the same percent survival with UVA (c′):

$$Dose {Effect}_{n} = \frac{\begin{matrix}( {Dose}^{′} ( - UVA ) to give survival n / \\ Dose ( + UVA ) to give survial n ) - 1\end{matrix}}{\begin{matrix}( Dose ( - UVA ) to give survival n / \\ Dose ( + UVA ) to give survival n ) + 1\end{matrix}}$$

As the ratio increases, the Dose Effect term approaches 1.

In the example in FIG. 3, the Dose Effect is calculated for one point. The dose of 0.4 dose units is required to reduce cell viability (termed response on the y axis) to 66% in the absence of light while only 0.16 dose units are required to similarly reduce viability in the presence of light. The dose effect for 0.4 dose units is:

$${DE}_{0.4} = \frac{\vert ( 0.4 / 0.16 ) - 1 \vert}{\vert ( 0.4 / 0.16 ) + 1 \vert} = 0.43$$

The Response Effect at dose c compares the percent survival with and without UVA at that dose and normalizes for the total range of the response over the range of doses evaluated (n₁ to nᵢ).

$$Response {Effect}_{c} = \frac{R ( - UVA ) c - R ( + UVA ) c}{{R}_{0}}$$

where R₀ is the Total Survival Range (up to 1000%), R(−UVA)c is the survival without UVA at dose c, and R(+UVA)c is the survival with UVA at dose c.

As the difference between the survival without UVA at dose c and the survival with UVA at dose c [ie., R(−UVA)c−R(+UVA)c] increases (indicative of phototoxic potential), then the Response Effect_{c} approaches 1.0.

Again in FIG. 3, the Response Effect for the 0.4 dose is:
RE_{0.4}=(66%−11%)/100%=0.55
The PE in this example is PE_{0.4}=0.43*0.55=0.24

The Mean Photo Effect is the mean of the individual Photo Effect values over the range evaluated. It is produced from the formula:

$$MPE = \frac{{\sum }_{i = 1}^{n} {w}_{i} * {PE}_{ci}}{{\sum }_{i = 1}^{n} {w}_{i}}$$

were wᵢ is a weighting factor for the highest viability observed for each curve.

The MPE value is used to determine phototoxic potential. In the original analysis of the validation data, a material was considered nonphototoxic if the MPE was <0.1 (this includes negative MPE values) and phototoxic if the MPE was ≧0.1 (Spielmann et al, 1998). This cut off was re-examined once the software had been rewritten and the weighting factor added. In the draft Organization for Economic Cooperation and Development phototoxicity test guideline (Guideline 432), MPE values of <0.1 (including negative values) are considered indicative of a nonphototoxin, values of 0.1 to <0.15 are considered probable phototoxins, and greater than and equal to 0.15 clear phototoxins. This guideline is expected to become the standard after final approval in 2003. The software used to calculate the MPE values is part of this guideline.

The following table shows the phototoxicity values for several tetracycline derivatives. The positive control is chlorpromazine. The phototoxicity is evaluated in terms of MPE and in terms of PIF using the new OECD draft guideline.

### Example 38

The following example demonstrates a response of selected fungi to CMT-3, 4, 7, 8, and the following derivivatives of CMT-3: 302, 303, 306, 308, 309 and 315.

The following fungi were inoculated onto potato dextrose agar (PDA) from stock cultures and incubated aerobically at 30° C.: Aspergillus fumigatus ATCC 1022, Penicillium sp. (laboratory isolate), Candida albicans, ATCC 14053, and Rhizopus sp.

A sterile cotton tipped applicator was moistened with sterile 0.9% saline and rolled over the surface of PDA slants of Aspergillus fumigatus, Rhizopus sp. and Penicillium sp. which demonstrated copious conidiogenesis. The conidia were suspended in 0.9% saline and the turbidity was adjusted to match a 0.5 MacFarland standard (equivalent to approximately 1.5×10⁸ cells). Candida albicans was suspended in saline and adjusted to 0.5 MacFarland in a similar manner. These suspensions were diluted 1:100 in sterile 0.9% saline.

SABHI Agar (Difco) pH 7.0 was prepared in 100 ml amounts and sterilized at 121° C. for 15 min. After the SABHI agar base cooled to 50°, 10 ml of each of the CMT substances were prepared in 10% DMSO at a concentration of 250 μg/ml. The CMT substances were than added at a final concentration of 25 μg/ml of agar base.

SABHI Agar plates of each CMT and SAHBI agar without CMT using dimethylsulphoxide (DMSO) as a control were inoculated with 10 μl of conidia suspension of Aspergillus fumigatus, Penicillium sp. and Rhizopus sp. and 10 μl suspension of Candida albicans prepared as described above. The plates were then incubated aerobically for 24 hour and for 48 hours at 30° C.

The results are set forth in Table 1 (24 hr. incubation) and Table 2 (48 hr. incubation). The score table used for Tables 1 and 2 is set forth in Table 3.

TABLE 1
Growth at 25 μg/ml compared to control at 24 hrs incubation

| Organism | 3 | 4 | 7 | 8 | 302 | 303 | 306 | 308 | 309 | 315 | DMSO |
|---|---|---|---|---|---|---|---|---|---|---|---|
| Aspergillus Fumigatus | 0 | 0 | 0 | ± | 0 | ± | 1 | 0 | ± | 0 | 3 |
| Penicillium Sp. | 0 | 3 | 3 | 3 | 3 | 3 | 3 | 0 | 3 | 0 | 4 |
| Rhizopus sp. | 3 | 4 | 4 | 4 | 4 | 4 | 4 | 4 | 4 | 1 | 4 |
| Candida Albicans | 1 | 1 | 0 | 4 | 4 | 3 | 3 | 4 | 4 | 0 | 4 |

TABLE 2
Growth at 25 μg/ml compared to control at 48 hours

| Organism | 3 | 4 | 7 | 8 | 302 | 303 | 306 | 308 | 309 | 315 | DMSO |
|---|---|---|---|---|---|---|---|---|---|---|---|
| Aspergillus Fumigatus | 4 | 1 | 4 | 4 | 4 | 4 | 4 | 1 | 4 | 0 | 4 |
| Penicillium Sp. | 4 | 0 | 4 | 4 | 4 | 4 | 4 | 0 | 4 | 0 | 4 |
| Rhizopus sp. | 1 | 4 | 3 | 4 | 4 | 4 | 4 | 4 | 4 | 1 | 4 |
| Candida Albicans | 4 | 4 | 0 | 4 | 4 | 4 | 4 | 4 | 4 | 0 | 4 |

TABLE 3
Inhibition Score and Grading of Fungal Growth

| Growth | Inhibition | |
|---|---|---|
| Grade | Score | Description |
| 4 | 0% | Level of growth in the absence |
| | | of anti-fungal agent (control). |
| 3 | 25% | 25% reduction in growth of |
| | | colonies compared to control. |
| 2 | 50% | 50% reduction in growth of |
| | | colonies compared to control. |
| 1 | 75% | 75% reduction in growth in |
| | | colonies compared to control. |
| 0 | 100% | complete inhibition of growth. |

CMT-315 yielded the best results with activity against all the fungi tested. CMT-308 demonstrated activity against Aspergillus fumigatus and Penicillium sp. CMT-4 demonstrated activity against Penicillium sp., and Aspergillus f. CMT-7 demonstrated strong activity against Candida albicans. CMT-3 inhibited Rhizopus sp., which is the most rapidly growing of the fungi, and can cause Rhinocerebral infection, pulnonary infection, mycotic keratitis, intraocular infection, orbital cellulitis, deep wound infection, external otomycosis, dermatitis, etc.

### Example 39

This example demonstrates a direct comparison between CMT-3 and Amphotericin B (AmB), a conventional anti-fungal agent, in the inhibition of Aspergillus f. The plates were prepared as described above, using 0.125, 0.5, 0.50, 1.00 and 2.00 concentrations of each of the drugs tested. DMSO was used as a control

The results are shown in Table 4 below. The results were graded according to the criteria set forth in Table 3.

TABLE 4

| | Conc. (μg/ml) | | | | |
|---|---|---|---|---|---|
| | 0.125 | 0.25 | 0.50 | 1.00 | 2.00 |
| CMT-3 | 4 | 2 | 1 | ±0 | 0 |
| AmB | 4 | 2 | 1 | 0 | 0 |

The results demonstrate that at various concentrations, the CMT-3 inhibited growth of Aspergillus f. as effective as AmB. At a concentration of 1.0 μg/ml, AmB inhibited 100% of fungal growth, while CMT-3 inhibited 95% of growth. At 2.0 μg/ml, both AmB and CMT-3 inhibited 100% of growth. Importantly, unlike AmB, CMT-3 demonstrates very little toxicity in vivo at 2.0 μg/ml concentration.

### Example 40

This example demonstrates the concentration of anti-fungal agent required to reduce the growth of the fungus by 50% in vitro (IC50) and the minimum concentration required to completely inhibit the growth of the fungus in vitro (MIC). CMTs utilized in the method of the invention, i.e CMT-3 and CMT-8 were compared to Doxycycline and Amphotericin B on microplate agar gels.

Each drug was dissolved in DMSO (1.0 mg/ml) as a stock solution and stored at −20° C. Just prior to use, each stock solution was thawed and diluted in DMSO to produce 6 different 100× concentrations. Potato dextrose agar was dissolved in distilled water (39 g/L) and sterilized at 138° C. (250° F.) for 15 min. The agar solution was mixed with each drug (in a water bath at 60° C.) to make a series of final concentrations, i.e. 0.00, 0.25, 0.50, 1.00, 2.00, 4.00 μg/ml. The mixtures were then transferred to 24-well plates (1 ml/well). After the gel had formed, the fungus in PBS (spore count=1-5×10⁴/ml) was inoculated by pipetting 10 μl onto each gel. The plates were incubated at 30° C. for different times, depending on the requirement of each species, e.g. 24 hours for Penicillium, Rhizopus, Tricothecium, Ulocladium, Absidia, Aspergilus, Caindida, Cunninghamella, 3 days for Scedosporium, and 5 days for Fonsecae and Phialophora.

The MICs and IC50s for the 11 different fungi are set forth in Table 5. “*” indicates better than or similar results to Amphotericin B. “NI” indicates no detectable inhibition.

TABLE 5

| | IC50(μg/ml) | MIC(μg/ml) |
|---|---|---|
| Candida Albicans | | |
| AmB | 0.5 | 1.0 |
| CMT-3 | 1.0 | 2.0 |
| CMT-8 | NI | NI |
| Doxy | | |
| | NI | NI |
| Rhizopus Species | | |
| AmB | 0.4 | 1.0 |
| CMT-3 | 0.8 | 2.0 |
| CMT-8 | NI | NI |
| Doxy | NI | NI |
| Aspergilus Fumigatus | | |
| AmB | 0.8 | 2.0 |
| CMT-3 | 0.5 | 1.0* |
| CMT-8 | NI | NI |
| Doxy | NI | NI |
| Penicillium Species | | |
| AmB | 0.12 | 0.25 |
| CMT-3 | 0.2 | 0.5 |
| CMT-8 | 2.0 | >4 |
| Doxy | NI | NI |
| Absidia Species | | |
| AmB | 1.0 | 4.0 |
| CMT-3 | 1.5 | 4.0* |
| CMT-8 | NI | NI |
| Doxy | NI | NI |
| Scedosporium Apiospermum | | |
| AmB | 4.0 | >4 |
| CMT-3 | 0.2 | 1.5* |
| CMT-8 | 2.0 | >4 |
| Doxy | NI | NI |
| Phialophora Verrucosa | | |
| AmB | NI | NI |
| CMT-3 | 1.5 | 4.0* |
| CMT-8 | NI | NI |
| Doxy | NI | NI |
| Cunninghamella Species | | |
| AmB | NI | NI |
| CMT-3 | 2.0 | 4.0* |
| CMT-8 | NI | NI |
| Doxy | NI | NI |
| Tricothecium Species | | |
| AmB | NI | NI |
| CMT-3 | 0.2 | 1.5* |
| CMT-8 | 0.7 | 2.0 |
| Doxy | 4.0 | >4 |
| Ulocladium Species | | |
| AmB | 1.0 | 2.0 |
| CMT-3 | 0.25 | 1.0* |
| CMT-8 | 2.0 | >4 |
| Doxy | NI | NI |
| Fonsecae Species | | |
| AmB | 4.0 | >4.0 |
| CMT-3 | 1.0 | 4.0* |
| CMT-8 | NI | NI |
| Doxy | NI | NI |

Thus, CMT-3 was effective on all 11 tested fungi, and CMT-8 had effects on some of these fungi. However, for 8 fungi out of the 11 different species of fungi, Amphotericin B showed the same or less antifungal activity than CMT-3. Doxy had essentially no detectable antifungal activity in this experiment.

### Example 41

This example demonstrates the antifungal activity of CMT-3 and Amphotericin B in vitro as being fungistatic (i.e. arresting the growth of the fungus) or fungicidal (i.e. killing the fungus).

In the pre-treatment phase of the experiment, Penicillium spores were suspended in PBS to achieve a spore count of 10⁷/ml. CMT-3 and Amphotericin B were dissolved in DMSO to reach a concentration of 1.0 mg/ml as stock solutions. 10 or 50 μl aliquots of these stock solutions were added to the incubation mixture (containing 1.0 ml of 10⁷/ml of Penicillium spores in PBS) to achieve a final concentration of 10 μg/ml or 50 μg/ml, respectively, for both drugs. The various incubations of Penicillium were carried out for 24 hours at 30° C.

After the pre-treatment phase, the reaction mixtures were diluted 1000 times with PBS, reducing the concentration of both drugs to 0.01 μg/ml or 0.05 μg/ml, and reducing the Penicillium spore count to 10⁴/ml. These drug concentrations of both CMT-3 and Amphotericin B would not be expected to inhibit the growth of the viable Penicillium spores.

Controls were then prepared. Before incubation, each tube was either not diluted further, or diluted to ½ or ¼ with PBS to produce tubes with three different spore counts, ie, 10⁴/ml, or 0.5×10⁴/ml, or 0.25×10⁴/ml. These cultures were then inoculated on potato dextrose agar gels in 24-well plates, and incubated at 30° C. for 48 hours to determine the rate of growth of the fungus as described before.

The controls were prepared from the suspension in the pre-treatment phase containing only Penicillium spores 10⁷/ml, and PBS. This control was diluted by 1000 times with PBS to produce a spore count of 10^{4/}ml. 1.0 ml of this diluted spore suspension was added to eight tubes. The stock solutions of CMT-3 and Amphotericin B, and DMSO were also diluted by 1000 times with PBS (the new concentration being 1.0 μg/ml for both drugs and 0.1% for DMSO), and 10 or 50 μl of these solutions was added into the above tubes. The final concentrations in each tube was either 0.01 μg/ml or 0.05 μg/ml for both drugs (CMT-3 or AmB), or 0.001% or 0.005% for DMSO. These tubes were further treated as described above to determine the growth of the fungus as controls.

The results demonstrated that all controls, including the concentration of 0.01 and 0.05 μl/ml of both drugs (CMT-3 and Amphotericin B), showed the same growth rate of Penicillium as the cultures without drugs, demonstrating that these low concentrations of both drugs did not inhibit the growth of the fungus in these control cultures.

Cultures of the Penicillium, after pretreatment with 10 and 50 μl/ml of Amphotericin B, showed the same rate of growth as PBS and DMSO controls during the subsequent incubation phase of the experiment, indicating that this drug did not kill the spores during the pre-treatment phase.

In contrast, cultures of Penicillium after pretreatment with 10 and 50 μl/ml of CMT-3, showed little or no growth on the agar gels compared with the controls, demonstrating that CMT-3 did kill the fungal spores during the pre-treatment phase.

Thus, Amphotericin B exhibited fungistatic activity, i.e. fungal growth was arrested but the fugal spores were not killed. On the other hand, CMT-3 exhibited fungicidal activity against Penicillium, killing the fungus.

PHOTOTOXICITY VALUES

| COMPOUND | MPE | PIF 1 | PIF 2 |
|---|---|---|---|
| Chloipromazine | 0.639 | N/D | 40.38 |
| Tetracycline | 0.340 | 5.38 | N/A |
| Doxycycline | 0.522 | 23.37 | 26.71 |
| Minocycline | 0.041 | 2.04 | N/A |
| COL 10 | 0.099 | 1.82 | 2.04 |
| COL 1 | 0.460 | N/D | N/A |
| COL 2 | 0.005 | N/D | N/A |
| COL 3 | 0.654 | 647 | 84.72 |
| COL 302 | 0.378 | 23.16 | 23.32 |
| COL 303 | 0.309 | 5.27 | 13.82 |
| COL 305 | 0.420 | N/D | N/A |
| COL 306 | 0.038 | 1.64 | 1.56 |
| COL 307 | 0.056 | 1.17 | N/A |
| COL 308 | 0.015 | 1.0 | N/A |
| COL 309 | 0.170 | 5.17 | 12.87 |
| COL 311 | 0.013 | 1.0 | N/A |
| COL 312 | 0.442 | 62.67 | 75.11 |
| COL 313 | 0.462 | 80.27 | 58.22 |
| COL 314 | 0.475 | 41.1 | 89.48 |
| COL 315 | 0.276 | 15.8 | 35.30 |
| COL 4 | 0.570 | N/D | N/A |
| COL 5 | 0.186 | N/D | N/A |
| COL 6 | 0.155 | N/D | N/A |
| COL 7 | 0.531 | N/D | N/A |
| COL 8 | 0.703 | 165 | 82.61 |
| COL 801 | −0.001 | 1.0 | N/A |
| COL 802 | −0.123 | 1.0 | N/A |
| COL 803 | 0.047 | N/D | N/A |
| COL 804 | 0.003 | 1.0 | N/A |
| COL 805 | 0.022 | 1.0 | N/A |
| COL 807 | 0.382 | 40.4 | N/A |
| COL 808 | 0.387 | 46.45 | N/A |
| COL 809 | 0.420 | N/D | N/A |
| COL 9 | 0.546 | N/D | N/A |
| COL 1001 | 0.025 | N/D | N/A |
| COL 1002 | 0.040 | 1.0 | 1.35 |

N/A indicates that the IC₅₀ value could not be determined for the UVA exposed and/or non-exposed groups
N/D indicates that the PIF1 was not determined for the particular compound, or was N/A as defined above.

In the present specification, some of the compounds of the invention are referred to by codes names. The correspondence between the compound and codes names are as follows:

CHEMICAL NAMES OF THE COL COMPOUNDS

| | |
|---|---|
| COL-1 | 4-dedimethylaminotetracycline |
| COL-3 | 6-demethyl-6-deoxy-4-dedimethylaminotetracycline |
| COL-301 | 7-bromo-6-demethyl-6-deoxy-4-dedimethylaminotetracycline |
| COL-302 | 7-nitro-6-demethyl-6-deoxy-4-dedimethylaminotetracycline |
| COL-303 | 9-nitro-6-demethyl-6-deoxy-4-dedimethylaminotetracycline |
| COL-304 | 7-acetamido-6-demethyl-6-deoxy-4-dedimethylaminotetracycline |
| COL-305 | 9-acetamido-6-demethyl-6-deoxy-4-dedimethylaminotetracycline |
| COL-306 | 9-dimethylamino-6-demethyl-6-deoxy-4-dedimethylaminotetracycline |
| COL-307 | 7-amino-6-demethyl-6-deoxy-4-dedimethylaminotetracycline |
| COL-308 | 9-amino-6-demethyl-6-deoxy-4-dedimethylaminotetracycline |
| COL-309 | 9-dimethylaminoacetamido-6-demethyl-6-deoxy-4-dedimethylaminotetracycline |
| COL-310 | 7-dimethylamino-6-demethyl-6-deoxy-4-dedimethylaminotetracycline |
| COL-311 | 9-palmitamide-6-demethyl-6-deoxy-4-dedimethylaminotetracycline |
| COL-312 | 2-CONHCH₂-pyrrolidin-1-yl-6-demethyl-6-deoxy-4-dedimethylaminotetracycline |
| COL-313 | 2-CONHCH₂-piperidin-1-yl-6-demethyl-6-deoxy-4-dedimethylaminotetracycline |
| COL-314 | 2-CONHCH₂-morpholin-1-yl-6-demethyl-6-deoxy-4-dedimethylaminotetracycline |
| COL-315 | 2-CONHCH₂-piperazin-1-yl-6-demethyl-6-deoxy-4-dedimethylaminotetracycline |
| COL-4 | 7-chloro-4-dedimethylaminotetracycline |
| COL-5 | tetracycline pyrazole |
| COL-6 | 4-hydroxy-4-dedimethylaminotetracycline |
| COL-7 | 4-dedimethylamino-12α-deoxytetracycline |
| COL-8 | 4-dedimethylaminodoxycycline |
| COL-801 | 9-acetamido-4-dedimethylaminodoxycycline |
| COL-802 | 9-dimethylaminoacetamido-4-dedimethylaminodoxycycline |
| COL-803 | 9-palmitamide-4-dedimethylaminodoxycycline |
| COL-804 | 9-nitro-4-dedimethylaminodoxycycline |
| COL-805 | 9-amino-4-dedimethylaminodoxycycline |
| COL-806 | 9-dimethylamino-4-dedimethylaminodoxycycline |
| COL-807 | 2-CONHCH₂-pyrrolidin-1-yl-4-dedimthylaminodoxycycline |
| COL-808 | 2-CONHCH₂-piperidin-1-yl-4-dedimethylaminodoxycycline |
| COL-809 | 2-CONHCH₂-piperazin-1-yl-4-dedimethylaminodoxycycline |
| COL-10 | 4-dedimethylaminominocycline (a.k.a. COL-310) |
| COL-1001 | 7-trimethylammonium-4-dedimethylaminosancycline |
| COL-1002 | 9-nitro-4-dedimethylaminominocycline |

### Index Of Structure

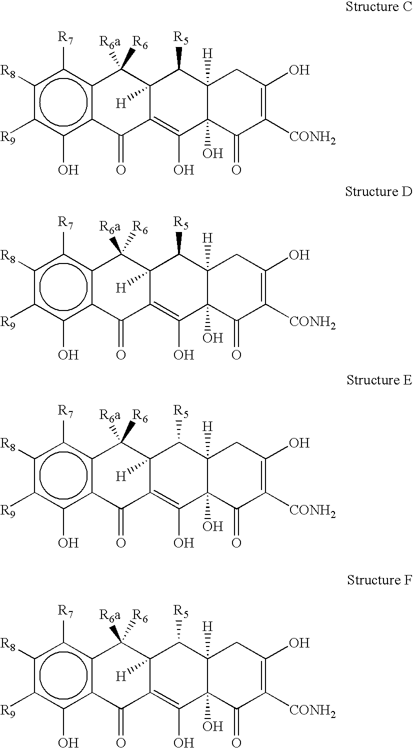 wherein R7 is selected from the group consisting of hydrogen, amino, nitro, mono(lower alkyl)amino, halogen, di(lower alkyl)amino, ethoxythiocarbonylthio, azido, acylamino, diazonium, cyano, and hydroxyl; R6-a is selected from the group consisting of hydrogen and methyl; R6 and R5 are selected from the group consisting of hydrogen and hydroxyl; R8 is selected from the group consisting of hydrogen and halogen; R9 is selected from the group consisting of hydrogen, amino, azido, nitro, acylamino, hydroxy, ethoxythiocarbonylthio, mono(lower alkyl)amino, halogen, diazonium, di(lower alkyl)amino and RCH(NH₂)CO; R is hydrogen or lower alkyl; and pharmaceutically acceptable and unacceptable salts thereof; with the following provisos: when either R7 and R9 are hydrogen then R8 must be halogen; and when R6-a, R6, R5 and R9 are all hydrogen and R7 is hydrogen, amino, nitro, halogen, dimethylamino or diethylamino, then R8 must be halogen; and when R6-a is methyl, R6 and R9 are both hydrogen, R5 is hydroxyl and R7 is hydrogen, amino, nitro, halogen or diethylamino, then R8 is halogen; and when R6-a is methyl, R6 is hydroxyl, R5, R7 and R9 are all hydrogen, then R8 must be halogen; and when R6-a, R6 and R5 are all hydrogen, R9 is methylamino and R7 is dimethylamino, then R8 must be halogen; and when R6-a is methyl, R6 is hydrogen, R5 is hydroxyl, R9 is methylamino and R7 is dimethylamino, then R8 must be halogen; and when R6-a is methyl, R6, R5 and R9 are all hydrogen and R7 is cyano, then R8 must be halogen. 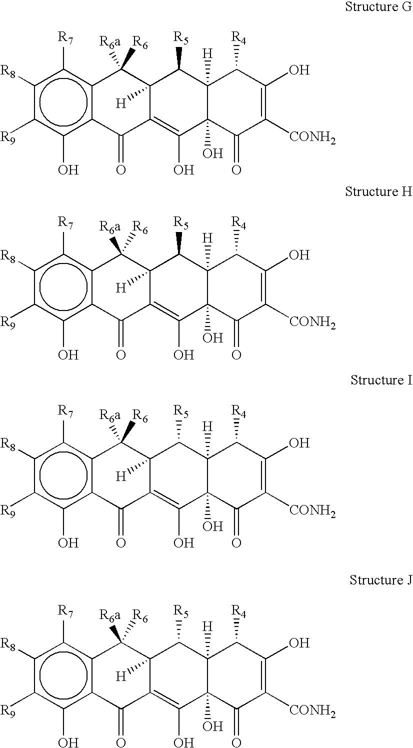
wherein R7 is selected from the group consisting of hydrogen, amino, nitro, mono(lower alkyl)amino, halogen, di(lower alkyl)amino, ethoxythiocarbonylthio, azido, acylamino, diazonium, cyano, and hydroxyl; R6-a is selected from the group consisting of hydrogen and methyl; R6 and R5 are selected from the group consisting of hydrogen and hydroxyl; R4 is selected from the group consisting of NOH, N—NH-A, and NH-A, where A is a lower alkyl group; R8 is selected from the group consisting of hydrogen and halogen; R9 is selected from the group consisting of hydrogen, amino, azido, nitro, acylamino, hydroxy, ethoxythiocarbonylthio, mono(lower alkyl)amino, halogen, di(lower alkyl)amino and RCH(NH₂)CO; R is hydrogen or lower alkyl; and pharmaceutically acceptable and unacceptable salts thereof; with the following provisos: when R4 is NOH, N—NH-alkyl or NH-alkyl and R7, R6-a, R6, R5, and R9 are all hydrogen, then R8 must be halogen; and when R4 is NOH, R6-a is methyl, R6 is hydrogen or hydroxyl, R7 is halogen, R5 and R9 are both hydrogen, then R8 must be halogen; and when R4 is N—NH-alkyl, R6-a is methyl, R6 is hydroxyl and R7, R5, R9 are all hydrogen, then R8 must be halogen; and when R4 is NH-alkyl, R6-a, R6, R5 and R9 are all hydrogen, R7 is hydrogen, amino, mono(lower alkyl)amino, halogen, di(lower alkyl)amino or hydroxyl, then R8 must be halogen; and when R4 is NH-alkyl, R6-a is methyl, R6 and R9 are both hydrogen, R5 is hydroxyl, and R7 is mono(lower alkyl)amino or di(lower alkyl)amino, then R8 must be halogen; and when R4 is NH-alkyl, R6-a is methyl, R6 is hydroxy or hydrogen and R7, R5, and R9 are all be hydrogen, then R8 must be halogen.

### General Formula (I)

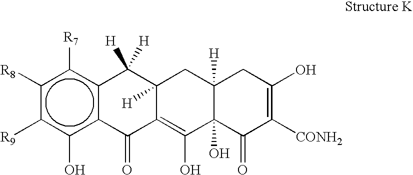

wherein R7, R8, and R9 taken together in each case, have the following meanings:

| | | |
|---|---|---|
| R7 | R8 | R9 |

| | | |
|---|---|---|
| azido | hydrogen | hydrogen |
| dimethylamino | hydrogen | azido |
| hydrogen | hydrogen | amino |
| hydrogen | hydrogen | azido |
| hydrogen | hydrogen | nitro |
| dimethylamino | hydrogen | amino |
| acylamino | hydrogen | hydrogen |
| hydrogen | hydrogen | acylamino |
| amino | hydrogen | nitro |
| hydrogen | hydrogen | (N,N-dimethyl)glycylamino |
| amino | hydrogen | amino |
| hydrogen | hydrogen | ethoxythiocarbonylthio |
| dimethylamino | hydrogen | acylamino |
| dimethylamino | hydrogen | diazonium |
| dimethylamino | chloro | amino |
| hydrogen | chloro | amino |
| amino | chloro | amino |
| acylamino | chloro | acylamino |
| amino | chloro | hydrogen |
| acylamino | chloro | hydrogen |
| inonoalkylamino | chloro | amino |
| nitro | chloro | amino |
| dimethylamino | chloro | acylamino |
| dimethylamino | chloro | dimethylamino |
| dimethylamino | hydrogen | hydrogen |
| hydrogen | hydrogen | dimethylamino |
| and | | |

General Formula (II)

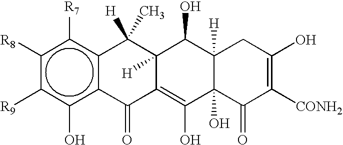

Structure L

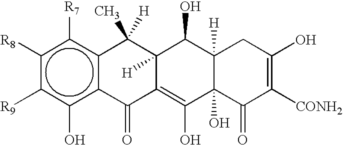

Structure M

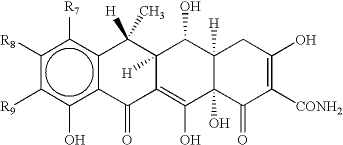

Structure N

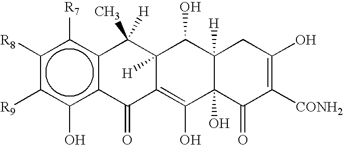

Structure O

wherein R7, R8, and R9 taken together in each case, have the following meanings:

| R7 | R8 | R9 |
|---|---|---|
| azido | hydrogen | hydrogen |
| dimethylamino | hydrogen | azido |
| hydrogen | hydrogen | amino |
| hydrogen | hydrogen | azido |
| hydrogen | hydrogen | intro |
| dimethylamino | hydrogen | amino |
| acylamino | hydrogen | hydrogen |
| hydrogen | hydrogen | acylamino |
| amino | hydrogen | nitro |
| hydrogen | hydrogen | (N,N-dimethyl)glycylamino |
| amino | hydrogen | amino |
| hydrogen | hydrogen | ethoxytbiocarbonyithic |
| dimethylamino | hydrogen | acylamino |
| hydrogen | hydrogen | diazonium |
| hydrogen | hydrogen | dirnethylamino |
| diazonium | hydrogen | hydrogen |
| ethoxythiocar- | hydrogen | hydrogen |
| bonylthio | | |
| dimethylamino | chloro | amino |
| amino | chloro | amino |
| acylamino | chloro | acylamino |
| hydrogen | chloro | ammo |
| amino | chloro | hydrogen |
| acylamino | chloro | hydrogen |
| monoalkyl amino | chloro | amino |
| nitro | chloro | amino |
| and | | |

General Formula (III)

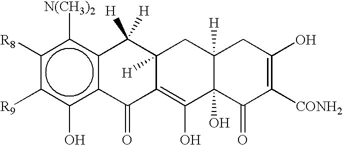

Structure P

wherein R8 is hydrogen or halogen and R9 is selected from the group consisting of nitro, (N,N-dimethyl)glycylamino, and ethoxythiocarbonylthio; and

### General Formula (IV)

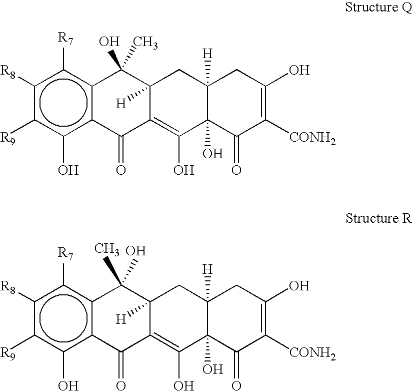

wherein R7, R8, and R9 taken together in each case, have the following meanings:

| R7 | R8 | | R9 |
|---|---|---|---|
| amino | hydrogen | | hydrogen |
| nitro | hydrogen | | hydrogen |
| azido | hydrogen | | hydrogen |
| dimethylamino | hydrogen | | azido |
| hydrogen | hydrogen | | amino |
| hydrogen | hydrogen | | azido |
| hydrogen | hydrogen | | nitro |
| bromo | hydrogen | | hydrogen |
| dimethylamino | hydrogen | | amino |
| acylamino | hydrogen | | hydrogen |
| hydrogen | hydrogen | | acylamino |
| amino | hydrogen | | nitro |
| hydrogen | hydrogen | | (N,N-dimethyl)glycylamino |
| amino | hydrogen | | amino |
| diethylamino | hydrogen | | hydrogen |
| hydrogen | hydrogen | | ethoxythiocarbonylthio |
| dimethylamino | hydrogen | | methylamino |
| dimethylamino | hydrogen | | acylamino |
| dimethylamino | chloro | | amino |
| amino | chloro | | amino |
| acylamino | chloro | | acylamino |
| hydrogen | chloro | | amino |
| amino | chloro | | hydrogen |
| acylamino | chloro | | hydrogen |
| monoalkylamino | chloro | | amino |
| nitro | chloro | | amino |
| hydrogen | hydrogen | | amino |
| hydrogen | hydrogen | | palmitamide |
| and | | | |
| STRUCTURE L | STRUCTURE M | STRUCTURE N | STRUCTURE O |

and pharmaceutically acceptable and unacceptable salts thereof. 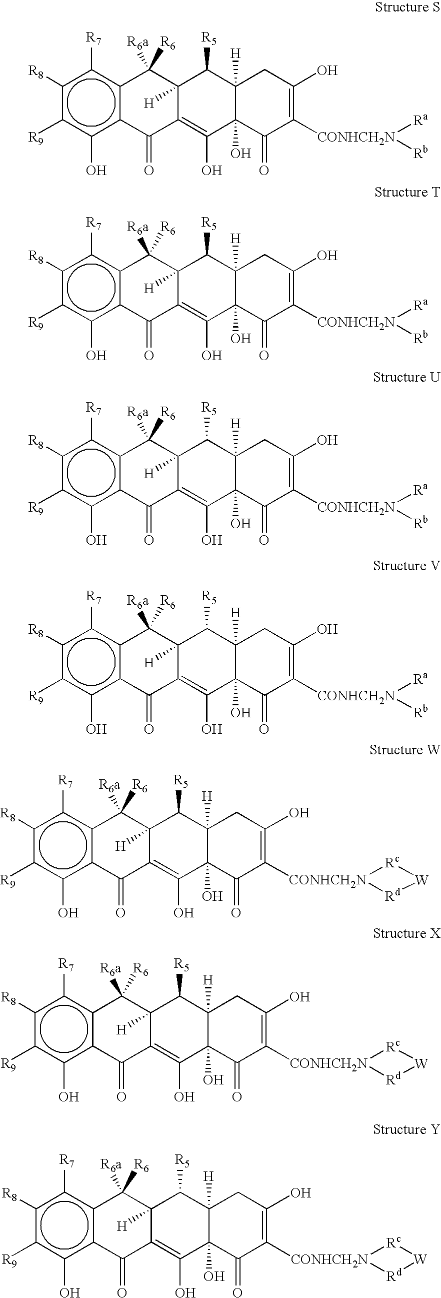 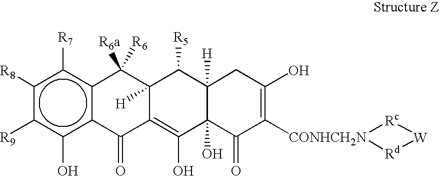
wherein R7 is selected from the group consisting of hydrogen, amino, nitro, mono(lower alkyl)amino, halogen, di(lower alkyl)amino, ethoxythiocarbonylthio, azido, acylamino, diazonium, cyano, and hydroxyl; R6-a is selected from the group consisting of hydrogen and methyl; R6 and R5 are selected from the group consisting of hydrogen and hydroxyl; R8 is selected from the group consisting of hydrogen and halogen; R9 is selected from the group consisting of hydrogen, amino, azido, nitro, acylamino, hydroxy, ethoxythiocarbonylthio, mono(lower alkyl)amino, halogen, diazonium, di(lower alkyl)amino and RCH(NH₂)CO; R is hydrogen or lower alkyl; R^{a} and R^{b} are selected from the group consisting of hydrogen, methyl, ethyl, n-propyl and 1-methylethyl with the proviso that R^{a} and R^{b} cannot both be hydrogen; R^{c} and R^{d} are, independently (CH₂)ₙCHR^{e} wherein n is 0 or 1 and R^{e} is selected from the group consisting of hydrogen, alkyl, hydroxy, lower(C₁-C₃) alkoxy, amino, or nitro; and, W is selected from the group consisting of (CHR^{e})ₘ wherein m is 0-3 and R^{e} is as above, NH, N(C₁-C₃) straight chained or branched alkyl, O, S and N(C₁-C₄) straight chain or branched alkoxy; and pharmaceutically acceptable and unacceptable salts thereof. In a further embodiment, the following provisos apply: when either R7 and R9 are hydrogen then R8 must be halogen; and when R6-a, R6, R5 and R9 are all hydrogen and R7 is hydrogen, amino, nitro, halogen, dimethylamino or diethylamino, then R8 must be halogen; and when R6-a is methyl, R6 and R9 are both hydrogen, R5 is hydroxyl, and R7 is hydrogen, amino, nitro, halogen or diethylamino, then R8 is halogen; and when R6-a is methyl, R6 is hydroxyl, R5, R7 and R9 are all hydrogen, then R8 must be halogen; and when R6-a, R6 and R5 are all hydrogen, R9 is methylamino and R7 is dimethylamino, then R8 must be halogen; and when R6-a is methyl, R6 is hydrogen, R5 is hydroxyl, R9 is methylamino and R7 is dimethylamino, then R8 must be halogen; and when R6-a is methyl, R6, R5 and R9 are all hydrogen and R7 is cyano, then R8 must be halogen.

### Structure K

wherein: R7, R8, and R9 taken together in each case, have the following meanings:

| | | |
|---|---|---|
| R7 | R8 | R9 |

| hydrogen | hydrogen | | amino |
|---|---|---|---|
| hydrogen | hydrogen | | palmitamide |
| and | | | |
| STRUCTURE L | STRUCTURE M | STRUCTURE N | STRUCTURE O |

wherein: R7, R8, and R9 taken together in each case, have the following meanings:

| R7 | R8 | R9 |
|---|---|---|
| hydrogen | hydrogen | acetamido |
| hydrogen | hydrogen | dimethylaminoacetamido |
| hydrogen | hydrogen | nitro |
| hydrogen | hydrogen | amino |
| and | | |

STRUCTURE P

wherein: R8, and R9 taken together are, respectively, hydrogen and nitro.

### Structure K

wherein: R7, R8, and R9 taken together are, respectively, hydrogen, hydrogen and dimethylamino.

| | | | |
|---|---|---|---|
| STRUCTURE | STRUCTURE | STRUCTURE | STRUCTURE |
| C | D | E | F |

wherein R7 is selected from the group consisting of an aryl, alkenyl and alkynyl; R6-a is selected from the group consisting of hydrogen and methyl; R6 and R5 are selected from the group consisting of hydrogen and hydroxyl; R8 is selected from the group consisting of hydrogen and halogen; R9 is selected from the group consisting of hydrogen, amino, azido, nitro, acylamino, hydroxy, ethoxythiocarbonylthio, mono(lower alkyl)amino, halogen, diazonium, di(lower alkyl)amino and RCH(NH₂)CO; and pharmaceutically acceptable and unacceptable salts thereof;

or

| | | | |
|---|---|---|---|
| STRUCTURE | STRUCTURE | STRUCTURE | STRUCTURE |
| C | D | E | F |

wherein: R7 is selected from the group consisting of hydrogen, amino, nitro, mono(lower alkyl)amino, halogen, di(lower alkyl)amino, ethoxythiocarbonylthio, azido, acylamino, diazonium, cyano, and hydroxyl; R6-a is selected from the group consisting of hydrogen and methyl; R6 and R5 are selected from the group consisting of hydrogen and hydroxyl; R8 is selected from the group consisting of hydrogen and halogen; R9 is selected from the group consisting of an aryl, alkenyl and alkynyl; and pharmaceutically acceptable and unacceptable salts thereof;

or

| | | | |
|---|---|---|---|
| STRUCTURE | STRUCTURE | STRUCTURE | STRUCTURE |
| C | D | E | F |

wherein: R7 and R9 are selected from the group consisting of an aryl, alkene, alkyne, or mixures thereof; R6-a is selected from the group consisting of hydrogen and methyl; R6 and R5 are selected from the group consisting of hydrogen and hydroxyl; R8 is selected from the group consisting of hydrogen and halogen; and pharmaceutically acceptable and unacceptable salts thereof.

| | | | |
|---|---|---|---|
| STRUCTURE | STRUCTURE | STRUCTURE | STRUCTURE |
| G | H | I | J |

wherein R7 is selected from the group consisting of an aryl, alkenyl and alkynyl; R6-a is selected from the group consisting of hydrogen and methyl; R6 and R5 are selected from the group consisting of hydrogen and hydroxyl; R4 is selected from the group consisting of NOH, N—NH-A, and NH-A,where A is a lower alkyl group; R8 is selected from the group consisting of hydrogen and halogen;R9 is selected from the group consisting of hydrogen, amino, azido, nitro, acylamino, hydroxy, ethoxythiocarbonylthio, mono(lower alkyl)amino, halogen, di(lower alkyl)amino and RCH(NH₂)CO; and pharmaceutically acceptable and unacceptable salts thereof;

or

| | | | |
|---|---|---|---|
| STRUCTURE | STRUCTURE | STRUCTURE | STRUCTURE |
| G | H | I | J |

wherein R7 is selected from the group consisting of hydrogen, amino, nitro, mono(lower alkyl)amino, halogen, di(lower alkyl)amino, ethoxythiocarbonylthio, azido, acylamino, diazonium, cyano, and hydroxyl; R6-a is selected from the group consisting of hydrogen and methyl; R6 and R5 are selected from the group consisting of hydrogen and hydroxyl; R4 is selected from the group consisting of NOH, N—NH-A, and NH-A, where A is a lower alkyl group; R8 is selected from the group consisting of hydrogen and halogen; R9 is selected from the group consisting of an aryl, alkenyl and alkynyl; and pharmaceutically acceptable and unacceptable salts thereof;

or

| | | | |
|---|---|---|---|
| STRUCTURE | STRUCTURE | STRUCTURE | STRUCTURE |
| G | H | I | J |

wherein: R7 and R9 are selected from the group consisting of an aryl, alkenyl, alkynyl; or mixtures thereof; R6-a is selected from the group consisting of hydrogen and methyl; R6 and R5 are selected from the group consisting of hydrogen and hydroxyl; R4 is selected from the group consisting of NOH, N—NH-A, and NH-A, where A is a lower alkyl group; and R8 is selected from the group consisting of hydrogen and halogen; and pharmaceutically acceptable and unacceptable salts thereof.

### Structure K

wherein R7 is selected from the group consisting of an aryl, alkenyl and alkynyl; R8 is selected from the group consisting of hydrogen and halogen; R9 is selected from the group consisting of hydrogen, amino, azido, nitro, acylamino, hydroxy, ethoxythiocarbonylthio, mono(lower alkyl)amino, halogen, di(lower alkyl)amino and RCH(NH₂)CO; and pharmaceutically acceptable and unacceptable salts thereof;

or

### Struture K

wherein: R7 is selected from the group consisting of hydrogen, amino, nitro, mono(lower alkyl)amino, halogen, di(lower alkyl)amino, ethoxythiocarbonylthio, azido, acylamino, diazonium, cyano, and hydroxyl; R8 is selected from the group consisting of hydrogen and halogen; R9 is selected from the group consisting of an aryl, alkenyl and alkynyl; and pharmaceutically acceptable and unacceptable salts thereof;

or

### Structure K

wherein: R7 and R9 are selected from the group consisting of an aryl, alkenyl, alkynyl and mixtures thereof; and R8 is selected from the group consisting of hydrogen and halogen; and pharmaceutically acceptable and unacceptable salts thereof;

and

| | | | |
|---|---|---|---|
| STRUCTURE | STRUCTURE | STRUCTURE | STRUCTURE |
| L | M | N | O |

wherein: R7 is selected from the group consisting of an aryl, alkenyl and alkynyl; R8 is selected from the group consisting of hydrogen and halogen; and pharmaceutically acceptable and unacceptable salts thereof;

or

| | | | |
|---|---|---|---|
| STRUCTURE | STRUCTURE | STRUCTURE | STRUCTURE |
| L | M | N | O |

wherein R7 is selected from the group consisting of hydrogen, amino, nitro, mono(lower alkyl)amino, halogen, di(lower alkyl)amino, ethoxythiocarbonylthio, azido, acylamino, diazonium, cyano, and hydroxyl; R8 is selected from the group consisting of hydrogen and halogen; R9 is selected from the group consisting of an aryl, alkenyl and alkynyl; and pharmaceutically acceptable and unacceptable salts thereof;

or

| | | | |
|---|---|---|---|
| STRUCTURE | STRUCTURE | STRUCTURE | STRUCTURE |
| L | M | N | O |

wherein R7 is and R9 are selected from the group consisting of an aryl, alkenyl, alkynyl and mixtures thereof; R8 is selected from the group consisting of hydrogen and halogen; R9 is selected from the group consisting of hydrogen, amino, azido, nitro, acylamino, hydroxy, ethoxythiocarbonylthio, mono(lower alkyl)amino, halogen, di(lower alkyl)amino and RCH(NH₂)CO; and pharmaceutically acceptable and unacceptable salts thereof;
and

### Structure P

wherein R9 is selected from the group consisting of an aryl, alkenyl and alkynyl; and R8 is selected from the group consisting of hydrogen and halogen; and pharmaceutically acceptable and unacceptable salts thereof;

and

| | |
|---|---|
| STRUCTURE Q | STRUCTURE R |

wherein R7 is selected from the group consisting of an aryl, alkenyl and alkynyl; R8 is selected from the group consisting of hydrogen and halogen; R9 is selected from the group consisting of hydrogen, amino, azido, nitro, acylamino, hydroxy, ethoxythiocarbonylthio, mono(lower alkyl)amino, halogen, di(lower alkyl)amino and RCH(NH₂)CO; and pharmaceutically acceptable and unacceptable salts thereof;

or

| | |
|---|---|
| STRUCTURE Q | STRUCTURE R |

wherein R7 is selected from the group consisting of hydrogen, amino, nitro, mono(lower alkyl)amino, halogen, di(lower alkyl)amino, ethoxythiocarbonylthio, azido, acylamino, diazonium, cyano, and hydroxyl; R8 is selected from the group consisting of hydrogen and halogen; R9 is selected from the group consisting of an aryl, alkenyl and alkynyl; and pharmaceutically acceptable and unacceptable salts thereof;

or

| | |
|---|---|
| STRUCTURE Q | STRUCTURE R |

wherein R7 and R9 are selected from the group consisting of an aryl, alkenyl, alkynyl; and mixtures thereof; R8 is selected from the group consisting of hydrogen and halogen; and pharmaceutically acceptable and unacceptable salts thereof.

### Structure S-Z

wherein R7 is selected from the group consisting of an aryl, alkenyl and alkynyl; R6-a is selected from the group consisting of hydrogen and methyl; R6 and R5 are selected from the group consisting of hydrogen and hydroxyl; R8 is selected from the group consisting of hydrogen and halogen; R9 is selected from the group consisting of hydrogen, amino, azido, nitro, acylamino, hydroxy, ethoxythiocarbonylthio, mono(lower alkyl)amino, halogen, diazonium, di(lower alkyl)amino and RCH(NH₂)CO; R^{a} and R^{b} are selected from the group consisting of hydrogen, methyl, ethyl, n-propyl and 1-methylethyl with the proviso that R^{a} and R^{b} cannot both be hydrogen; R^{c} and R^{d} are, independently, (CH₂)ₙCHR^{e} wherein n is 0 or 1 and R^{e} is selected from the group consisting of hydrogen, alkyl, hydroxy, lower(C₁-C₃)alkoxy, amino, or nitro; and, W is selected from the group consisting of (CHR^{e})ₘ wherein m is 0-3 and said R^{e} is as above, NH, N(C₁-C₃) straight chained or branched alkyl, O, S and N(C₁-C₄) straight chain or branched alkoxy; and pharmaceutically acceptable and unacceptable salts thereof;

or

### Structure S-Z

wherein R7 is selected from the group consisting of hydrogen, amino, nitro, mono(lower alkyl)amino, halogen, di(lower alkyl)amino, ethoxythiocarbonylthio, azido, acylamino, diazonium, cyano, and hydroxyl; R6-a is selected from the group consisting of hydrogen and methyl; R6 and R5 are selected from the group consisting of hydrogen and hydroxyl; R8 is selected from the group consisting of hydrogen and halogen; R9 is selected from the group consisting of an aryl, alkenyl and alkynyl; R^{a} and R^{b} are selected from the group consisting of hydrogen, methyl, ethyl, n-propyl and 1-methylethyl with the proviso that R^{a} and R^{b} cannot both be hydrogen; R^{a} and R^{d} are, independently, (CH₂)ₙCHR^{e} wherein n is 0 or 1 and R^{e} is selected from the group consisting of hydrogen, alkyl, hydroxy, lower(C₁-C₃) alkoxy, amino, or nitro; and, W is selected from the group consisting of (CHR^{e})ₘ wherein m is 0-3 and said R^{e} is as above, NH, N(C₁-C₃) straight chained or branched alkyl, 0, S and N(C₁-C₄) straight chain or branched alkoxy; and pharmaceutically acceptable and unacceptable salts thereof;

or

### Structure S-Z

wherein: R7 and R9 are selected from the group consisting of an aryl, alkenyl, alkynyl and mixtures thereof; R6-a is selected from the group consisting of hydrogen and methyl; R6 and R5 are selected from the group consisting of hydrogen and hydroxyl; R8 is selected from the group consisting of hydrogen and halogen; R^{a} and R^{b} are selected from the group consisting of hydrogen, methyl, ethyl, n-propyl and 1-methylethyl with the proviso that R^{a} and R^{b} cannot both be hydrogen; R^{c} and R^{d} are, independently, (CH₂)ₙCHR^{e} wherein n is 0 or 1 and R^{e} is selected from the group consisting of hydrogen, alkyl, hydroxy, lower(C₁-C₃) alkoxy, amino, or nitro; and W is selected from the group consisting of (CHR^{e})ₘ wherein m is 0-3 and said R^{e} is as above, NH, N(C₁-C₃) straight chained or branched alkyl, O, S and N(C₁-C₄) straight chain or branched alkoxy; and pharmaceutically acceptable and unacceptable salts thereof.

Throughout this specification, the descriptions of some structures include the term “lower alkyl.” The term “lower alkyl” means an alkyl group comprising relatively few carbon atoms, for example, about one to ten carbon atoms. A preferred low end of this range is one, two, three, four or five carbon atoms; and a preferred high end of this range is six, seven, eight, nine or ten carbon atoms. Some examples of “lower alkyl” groups include methyl groups, ethyl groups, propyl groups, isopropyl groups, butyl groups, etc.

1. A method for simultaneously treating mucositis and fungal infection in a mammal in need thereof, said method comprising administering to said mammal an effective amount of an anti-mucositis and anti-fungal pharmaceutical composition consisting of a tetracycline compound in an amount that is effective to simultaneously treat mucositis and fungal infection, but has substantially no antibiotic activity.

2. A method according to claim 1 wherein said fungus is selected from the group consisting of Cryptococcus species, Candida albicans, Rhizopus species, Aspergillus fumigatus, Penicillium species, Absidia species, Scedosporium apiospermum, Phialophora verrucosa, Cunninghamella species, Tricothecium species, Ulocladium species, Fonsecae species, and combinations thereof.

3. A method according to claim 1 wherein said fungus is selected from the group consisting of Rhizopus species, Absidia species, Scedosporium apiospermum, Phialophora verrucosa, Cunninghamella species, Tricothecium species, Ulocladium species, Fonsecae species, or a combination thereof, and wherein said non-antibiotic tetracycline derivative is CMT-3.

4. A method according to claim 1 wherein said fungus is Aspergillus fumigatus, Penicillium species, Rhizopus species, Candida albicans, or a combination thereof, and wherein said non-antibiotic tetracycline derivative is CMT-315.

5. A method according to claim 1 wherein said fungus is Penicillium species and said non-antibiotic tetracycline derivative is CMT4.

6. A method according to claim 1 wherein said fungus is Candida albicans and said non-antibiotic tetracycline derivative is CMT-7.

7. A method according to claim 1 wherein said fungus is Aspergillus fumigatus, Penicillium species or a combination thereof, and said non-antibiotic is CMT-308.

8. A method according to claim 1 wherein said fungus is Penicilliuln species, Scedosporium apiospermum, Tricothecium species, Ulocladium species, or a combination thereof and said non-antibiotic tetracycline derivative is CMT-8.

9. A method according to claim 1 wherein said mammal is a human.

10. A method according to claim 1 wherein said treatment comprises administering said non-antibiotic tetracycline derivative systemically.

11. A method according to claim 10, wherein said systemic administration is oral administration, intravenous injection, intramuscular injection, subcutaneous administration, transdermal administration or intranasal administration.

12. A method according to claim 1, wherein said tetracycline compound is an antibiotic tetracycline compound administered in an amount which is 10-80% of the antibiotic amount.

13. A method according to claim 1, wherein said tetracycline compound is doxycycline administered twice a day in a dose of 20 mg.

14. A method according to claim 1, wherein said tetracycline compound is minocycline administered once a day in a dose of 38 mg.

15. A method according to claim 1, wherein said tetracycline compound is minocycline administered twice a day in a dose of 38 mg.

16. A method according to claim 1, wherein said tetracycline compound is minocycline administered three times a day in a dose of 38 mg.

17. A method according to claim 1, wherein said tetracycline compound is tetracycline administered twice a day in a dose of 60 mg/day.

18. A method according to claim 1, wherein said tetracycline compound is tetracycline administered three times a day in a dose of 60 mg/day.

19. A method according to claim 1, wherein said tetracycline compound is tetracycline administered four times a day in a dose of 60 mg/day.

20. A method according to claim 1, wherein said tetracycline compound is an antibiotic tetracycline compound administered in an amount which results in a serum concentration which is 10-80% of the minimum antibiotic serum concentration.

21. A method according to claim 1, wherein said tetracycline compound is doxycycline administered in an amount which results in a serum concentration which is 1.0 μg/ml.

22. A method according to claim 1, wherein said tetracycline compound is minocycline administered in an amount which results in a serum concentration which is 0.8 μg/ml.

23. A method according to claim 1, wherein said tetracycline compound is tetracycline administered in an amount which results in a serum concentration which is 0.5 μg/ml.

24. A method according to claim 12 or 20, wherein said antibiotic tetracycline compound is doxycycline, minocycline, tetracycline, oxytetracycline, chlortetracycline, demeclocycline or pharmaceutically acceptable salts thereof.

25. A method according to claim 24, wherein said antibiotic tetracycline compound is doxycycline.

26. A method according to claim 25, wherein said doxycycline is administered in an amount which provides a serum concentration in the range of about 0.1 to about 0.8 μg/ml.

27. A method according to claim 25, wherein said doxycycline is administered in an amount of 20 milligrams twice daily.

28. A method according to claim 26, wherein said doxycycline is administered by sustained release over a 24 hour period.

29. A method according to claim 28, where said doxcycline is administered in an amount of 40 milligrams.

30. A method according to claim 1, wherein said tetracycline compound is a non-antibiotic tetracycline compound.

31. A method according to claim 30, wherein said non-antibiotic tetracycline compound is:
4-de(dimethylamino)tetracycline (CMT-1),
tetracyclinonitrile (CMT-2),
6-demethyl-6-deoxy-4-de(dimethylamino)tetracycline (CMT-3),
4-de(dimethylamino)-7-chlorotetracycline (CMT-4),
tetracycline pyrazole (CMT-5)
4-hydroxy-4-de(dimethylamino)tetracycline (CMT-6),
4-de(dimethylamino)-12α-deoxytetracycline (CMT-7),
6-α-deoxy-5-hydroxy-4-de(dimethylamino)tetracycline (CMT-8),
4-de(dimethylamino)-12α-deoxyanhydrotetracycline (CMT-9), or
4-de(dimethylamino)minocycline (CMT-10).

32. A method according to claim 30, wherein the non-antibiotic tetracycline compound is selected from the group consisting of: 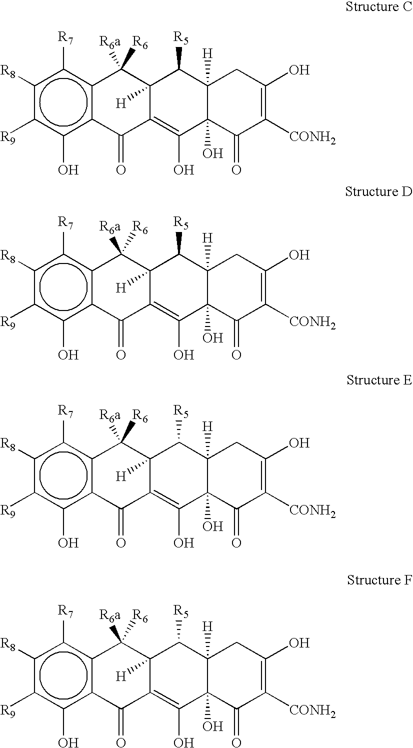
wherein:
R7 is selected from the group consisting of hydrogen, amino, nitro, mono(lower alkyl)amino, halogen, di(lower alkyl)amino, ethoxythiocarbonylthio, azido, acylamino, diazonium, cyano, and hydroxyl;
R6-a is selected from the group consisting of hydrogen and methyl;
R6 and R5 are selected from the group consisting of hydrogen and hydroxyl;
R8 is selected from the group consisting of hydrogen and halogen;
R9 is selected from the group consisting of hydrogen, amino, azido, nitro, acylamino, hydroxy, ethoxythiocarbonylthio, mono(lower alkyl)amino, halogen, diazonium, di(lower alkyl)amino and RCH(NH₂)CO;
R is hydrogen or lower alkyl; and pharmaceutically acceptable salts thereof; with the following provisos:
when either R7 and R9 are hydrogen then R8 must be halogen; and
when R6-a, R6, R5 and R9 are all hydrogen and R7 is hydrogen, amino, nitro, halogen, dimethylamino or diethylamino, then R8 must be halogen; and
when R6-a is methyl, R6 and R9 are both hydrogen, R5 is hydroxyl, and R7 is hydrogen, amino, nitro, halogen or diethylamino, then R8 is halogen; and
when R6-a is methyl, R6 is hydroxyl, R5, R7 and R9 are all hydrogen, then R8 must be halogen; and
when R6-a, R6 and R5 are all hydrogen, R9 is methylamino and R7 is dimethylamino, then R8 must be halogen; and
when R6-a is methyl, R6 is hydrogen, R5 is hydroxyl, R9 is methylamino and R7 is dimethylamino, then R8 must be halogen; and
when R6-a is methyl, R6, R5 and R9 are all hydrogen and R7 is cyano, then R8 must be halogen.

33. A method according to claim 30, wherein the non-antibiotic tetracycline compound is selected from the group consisting of: 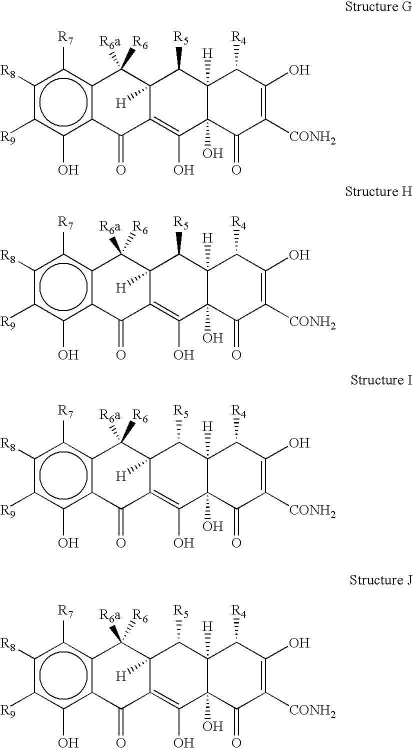
wherein:
R7 is selected from the group consisting of hydrogen, amino, nitro, mono(lower alkyl)amino, halogen, and di(lower alkyl)amino, ethoxythiocarbonylthio, azido, acylamino, diazonium, cyano, and hydroxyl;
R6-a is selected from the group consisting of hydrogen and methyl;
R6 and R5 are selected from the group consisting of hydrogen and hydroxyl;
R4 is selected from the group consisting of NOH, N—NH-A, and NH-A, where A is a lower alkyl group;
R8 is selected from the group consisting of hydrogen and halogen;
R9 is selected from the group consisting of hydrogen, amino, azido, nitro, acylamino, hydroxy, ethoxythiocarbonylthio, mono(lower alkyl)amino, halogen, di(lower alkyl)amino and RCH(NH₂)CO;
R is hydrogen or lower alkyl; and
pharmaceutically acceptable salts thereof; with the following provisos:
when R4 is NOH, N—NH-alkyl or NH-alkyl and R7, R6-a, R6, R5, and R9 are all hydrogen, then R8 must be halogen; and
when R4 is NOH, R6-a is methyl, R6 is hydrogen or hydroxyl, R7 is halogen, R5 and R9 are both hydrogen, then R8 must be halogen; and
when R4 is N—NH-alkyl, R6-a is methyl, R6 is hydroxyl and R7, R5, R9 are all hydrogen, then R8 must be halogen; and
when R4 is NH-alkyl, R6-a, R6, R5 and R9 are all hydrogen, R7 is hydrogen, amino, mono(lower alkyl)amino, halogen, di(lower alkyl)amino or hydroxyl, then R8 must be halogen; and
when R4 is NH-alkyl, R6-a is methyl, R6 and R9 are both hydrogen, R5 is hydroxyl, and R7 is mono(lower alkyl)amino or di(lower alkyl)amino, then R8 must be halogen; and
when R4 is NH-alkyl, R6-a is methyl, R6 is hydroxy or hydrogen and R7, R5, and R9 are all be hydrogen, then R8 must be halogen.

34. A method according to claim 30 wherein the non-antibiotic tetracycline compound is selected from the group consisting of: 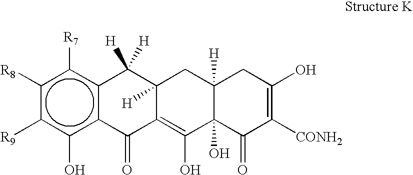
wherein: R7, R8, and R9 taken together in each case, have the following meanings:

| R7 | R8 | R9 |
|---|---|---|
| azido | hydrogen | hydrogen |
| dimethylamino | hydrogen | azido |
| hydrogen | hydrogen | amino |
| hydrogen | hydrogen | azido |
| hydrogen | hydrogen | nitro |
| dimethylamino | hydrogen | amino |
| acylamino | hydrogen | hydrogen |
| hydrogen | hydrogen | acylainino |
| amino | hydrogen | nitro |
| hydrogen | hydrogen | (N,N-dimethyl)glycylamino |
| amino | hydrogen | amino |
| hydrogen | hydrogen | ethoxytliiocarbonylthio |
| dimethylamino | hydrogen | acylamino |
| dimethylamino | hydrogen | diazonium |
| diniethylamino | chloro | amino |
| hydrogen | chloro | amino |
| amino | chloro | amino |
| acylainmo | chloro | acylamino |
| amino | chloro | hydrogen |
| acylamino | chloro | hydrogen |
| monoalkylamino | chloro | amino |
| nitro | chloro | amino |
| dimethylamino | chloro | acylamino |
| dimethylamino | chloro | dimethylamino |
| hydrogen | hydrogen | dimethylamino |
| dimethylamino | hydrogen | hydrogen |
| and | | |

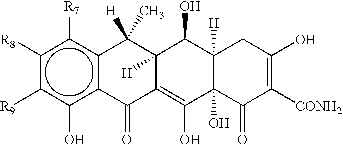

Structure L 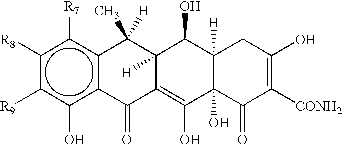
Structure M 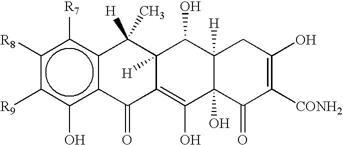
Structure N 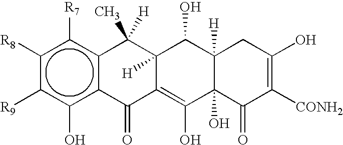
Structure O
wherein: R7, R8, and R9 taken together in each case, have the following meanings:

| R7 | R8 | R9 |
|---|---|---|
| azido | hydrogen | hydrogen |
| dimethylamino | hydrogen | azido |
| hydrogen | hydrogen | amino |
| hydrogen | hydrogen | azido |
| hydrogen | hydrogen | nitro |
| dimethylamino | hydrogen | amino |
| acylamino | hydrogen | hydrogen |
| hydrogen | hydrogen | acylamino |
| amino | hydrogen | intro |
| hydrogen | hydrogen | (N,N-dimethyl)glycylamino |
| amino | hydrogen | amino |
| hydrogen | hydrogen | ethoxythiocarbonylthio |
| dimethylamino | hydrogen | acylainino |
| hydrogen | hydrogen | diazonium |
| hydrogen | hydrogen | dimethylamino |
| diazonium | hydrogen | hydrogen |
| ethoxythiocar- | hydrogen | hydrogen |
| bonylthio | | |
| dimethylamino | chloro | amino |
| amino | chloro | ammo |
| acylamino | chloro | acylamino |
| hydrogen | chloro | ammo |
| amino | chloro | hydrogen |
| acylamino | chloro | hydrogen |
| monoalkylammo | chloro | amino |
| nitro | chloro | amino |
| and | | |

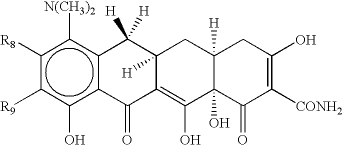

Structure P
wherein: R8 is hydrogen or halogen and R9 is selected from the group consisting of nitro, (N,N-dimethyl)glycylamino, and ethoxythiocarbonylthio; and 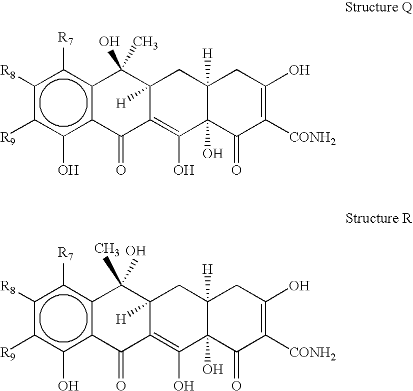
wherein: R7, R8, and R9 taken together in each case, have the following meanings:

| | | |
|---|---|---|
| R7 | R8 | R9 |
| amino | hydrogen | hydrogen |
| nitro | hydrogen | hydrogen |
| azido | hydrogen | hydrogen |
| dimethylamino | hydrogen | azido |
| hydrogen | hydrogen | amino |
| hydrogen | hydrogen | azido |
| hydrogen | hydrogen | nitro |
| bromo | hydrogen | hydrogen |
| dimethylamino | hydrogen | amino |
| acylamino | hydrogen | hydrogen |
| hydrogen | hydrogen | acylamino |
| amino | hydrogen | nitro |
| hydrogen | hydrogen | (N,N-dimethyl)glycylamino |
| amino | hydrogen | amino |
| diethylamino | hydrogen | hydrogen |
| hydrogen | hydrogen | ethoxythiocarbonylthio |
| dimethylamino | hydrogen | methylamino |
| dimethylamino | hydrogen | acylamino |
| dimethylamino | chloro | amino |
| amino | chloro | amino |
| acylamino | chloro | acylamino |
| hydrogen | chloro | amino |
| amino | chloro | hydrogen |
| acylamino | chloro | hydrogen |
| monoalkylamino | chloro | amino |
| nitro | chloro | amino |

and pharmaceutically acceptable salts thereof

35. A method according to claim 30, wherein the non-antibiotic tetracycline compound is selected from the group consisting of: 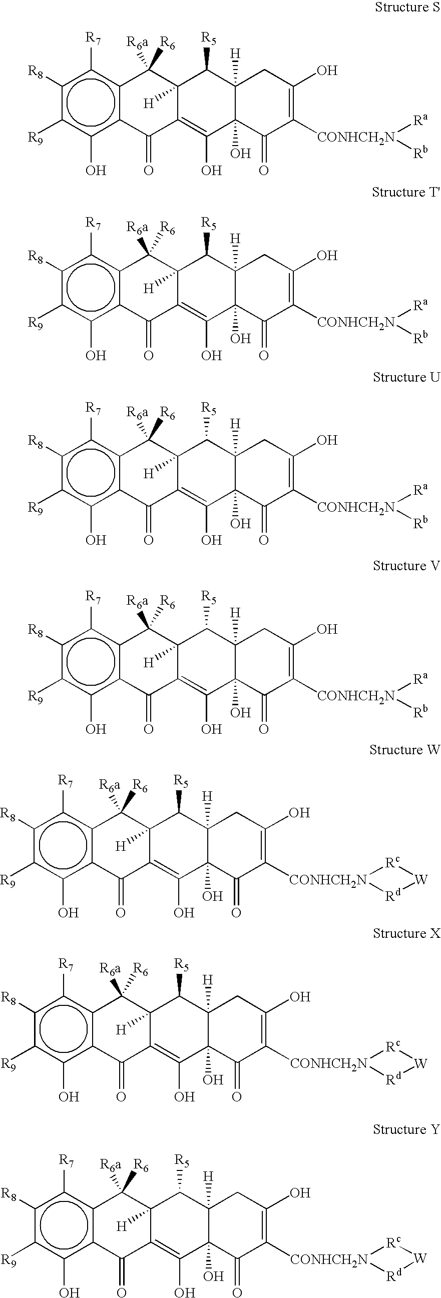 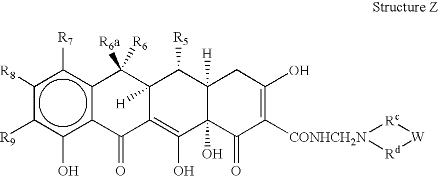
wherein:
R7 is selected from the group consisting of hydrogen, amino, nitro, mono(lower alkyl)amino, halogen, di(lower alkyl)amino, ethoxythiocarbonylthio, azido, acylamino, diazonium, cyano, and hydroxyl;
R6-a is selected from the group consisting of hydrogen and methyl;
R6 and R5 are selected from the group consisting of hydrogen and hydroxyl;
R8 is selected from the group consisting of hydrogen and halogen;
R9 is selected from the group consisting of hydrogen, amino, azido, nitro, acylamino, hydroxy, ethoxythiocarbonylthio, mono(lower alkyl)amino, halogen, diazonium, di(lower alkyl)amino and RCH(NH₂)CO;
R is hydrogen or lower alkyl;
R^{a} and R^{b} are selected from the group consisting of hydrogen, methyl, ethyl, n-propyl and 1-methylethyl with the proviso that R^{a} and R^{b} cannot both be hydrogen;
R^{c} and R^{d} are, independently, (CH₂)ₙCHR^{e} wherein n is 0 or 1 and R^{e} is selected from the group consisting of hydrogen, alkyl, hydroxy, lower(C₁-C₃) alkoxy, amino, or nitro; and,
W is selected from the group consisting of (CHR^{e})ₘ wherein m is 0-3 and said R^{e} is selected from the group consisting of hydrogen, alkyl, hydroxyl, lower(C₁-C₃), alkoxy, amino, nitro, NH, N(C₁-C₃) straight chained or branched alkyl, O, S and N(C₁-C₄) straight chain or branched alkoxy; and,
pharmaceutically acceptable salts thereof.

36. A method according to claim 35, wherein the non-antibiotic tetracycline compound selected from the group consisting of structures S-Z has the following provisos:
when either R7 and R9 are hydrogen then R8 must be halogen; and
when R6-a, R6, R5 and R9 are all hydrogen and R7 is hydrogen, amino, nitro, halogen, dimethylamino or diethylamino, then R8 must be halogen; and
when R6-a is methyl, R6 and R9 are both hydrogen, R5 is hydroxyl, and R7 is hydrogen, amino, nitro, halogen or diethylamino, then R8 is halogen; and
when R6-a is methyl, R6 is hydroxyl, R5, R7 and R9 are all hydrogen, then R8 must be halogen; and
when R6-a, R6 and R5 are all hydrogen, R9 is methylamino and R7 is dimethylamino, then R8 Pmust be halogen; and
when R6-a is methyl, R6 is hydrogen, R5 is hydroxyl, R9 is methylamino and R7 is dimethylamino, then R8 must be halogen; and
when R6-a is methyl, R6, R5 and R9 are all hydrogen and R7 is cyano, then R8 must be halogen.

37. A method according to claim 1, wherein said tetracycline compound has a photoirritancy factor of less than the photoirritancy factor of doxycycline.

38. A method according to claim 1, wherein said tetracycline compound has a photoirritancy factor from about one to about two.

39. A method according to claim 38, wherein said tetracycline compound has a general formula: 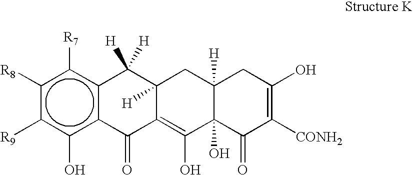
wherein R7, R8, and R9 taken together are, respectively, hydrogen, hydrogen and dimethylamino.

40. A method according to claim 1, wherein said tetracycline compound has a photoirritancy factor from about 1.0 to about 1.2.

41. A method according to claim 41, wherein said tetracycline compound is selected from the group consisting of: 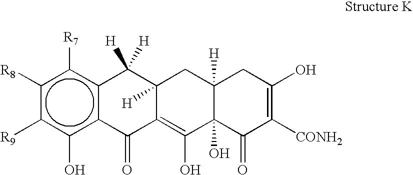
wherein R7, R8, and R9 taken together in each case, have the following meanings:

| R7 | R8 | R9 |
|---|---|---|
| hydrogen | hydrogen | amino |
| hydrogen | hydrogen | palmitamide |
| and | | |

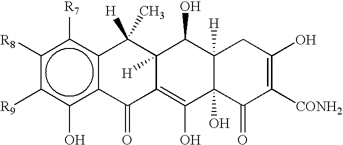

Structure L 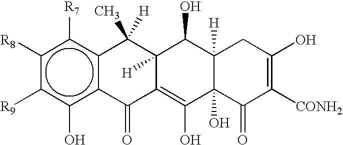
Structure M 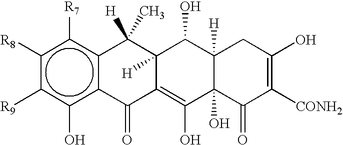
Structure N 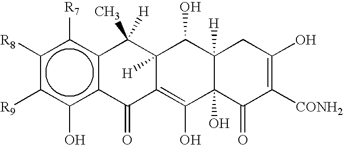
Structure O
wherein R7, R8, and R9 taken together in each case, have the following meanings:

| R7 | R8 | R9 |
|---|---|---|
| hydrogen | hydrogen | acetamido |
| hydrogen | hydrogen | dimethylaminoacetamido |
| hydrogen | hydrogen | nitro |
| hydrogen | hydrogen | amino |
| and | | |

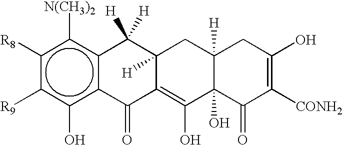

Structure P
wherein R8, and R9 taken together are, respectively, hydrogen and nitro.

42. A method according to claim 1 wherein said treatment comprises administering a non-antibiotic tetracycline derivative topically.

43. A method according to claim 42 wherein said non-antibiotic tetracycline derivative is administered in a mouthwash.
